(19) United States
(12) Patent Application Publication
(10) Pub. No.: US 2023/0137331 A1
(43) Pub. Date: May 4, 2023

(54) Activation and Expansion of T Cell Subsets Using Biocompatible Solid Substrates with Tunable Rigidity

(71) Applicants: THE TRUSTEES OF THE UNIVERSITY OF PENNSYLVANIA, Philadelphia, PA (US); THE TRUSTEES OF COLUMBIA UNIVERSITY IN THE CITY OF NEW YORK, New York, NY (US)

(72) Inventors: Michael C. Milone, Cherry Hill, NJ (US); Lance C. Kam, New York, NY (US); Keyue Shen, Cambridge, MA (US); Xueli Hao, Cleveland, OH (US); Roderick O'Connor, Philadelphia, PA (US); Geraldine Goebrecht, La Jolla, CA (US)

(21) Appl. No.: 17/839,397

(22) Filed: Jun. 13, 2022

Related U.S. Application Data
(63) Continuation of application No. 14/342,599, filed on Aug. 29, 2014, now abandoned.
(60) Provisional application No. 61/635,267, filed on Apr. 18, 2012.
(60) Provisional application No. 61/531,420, filed on Sep. 6, 2011.

(51) Int. Cl. A61K 35/17; C12N 5/0783; C07K 16/28

(52) U.S. Cl. CPC A61K 35/17; C12N 5/0636; C07K 16/2818; C07K 16/2809; C12N 2501/51; C12N 2527/00; C12N 2539/00; C12N 2501/515; C12N 2531/00; A61K 2035/124

(57) ABSTRACT

The present invention provides compositions and methods for activation and expansion of T cells using a biocompatible solid substrate with tunable rigidity. Rigidity of a substrate is an important parameter that can be used to control the overall expansion and differentiation of T cells.

### CROSS-REFERENCE TO RELATED APPLICATIONS

This application is a continuation of, and claims priority to, U.S. patent application Ser. No. 14/342,599, filed Aug. 29, 2014, which is the U.S. national phase application filed under 35 U.S.C. § 371 claiming benefit to International Patent Application No. PCT/US2012/053887, filed on Sep. 6, 2012, which is entitled to priority under 35 U.S.C. § 119(e) to U.S. Provisional Application No. 61/635,267, filed Apr. 18, 2012 and U.S. Provisional Application No. 61/531,420, filed Sep. 6, 2011, each of which are hereby incorporated by reference in their entireties.

### STATEMENT REGARDING FEDERALLY SPONSORED RESEARCH OR DEVELOPMENT

This invention was made with government support under grant number EY016586 awarded by the National Institutes of Health. The government has certain rights in the invention.

### BACKGROUND OF THE INVENTION

Adoptive immunotherapy holds great potential as a therapeutic modality for the treatment of a variety of diseases including cancer and chronic viral infections (June, 2007, J Clin Invest. 117(6):1466-76). Central to these therapeutic approaches are controllable platforms for ex vivo activation of T cells. Several cell-based and artificial substrate systems have been described (June, 2007, J Clin Invest. 117(5):1204-12). Agonist antibodies to CD3 and CD28 immobilized on rigid materials like polystyrene plastic, and glass, are widely used in many of these systems for the activation and expansion of T cells. These artificial culture substrates are also widely used in basic studies of T cell activation forming the foundation for much of our knowledge of T signal transduction (Kruisbeek A, Shevach E, Thornton A. Proliferative Assays for T Cell Function. In: Coligan J E, editor. Current protocols in immunology. New York: John Wiley and Sons).

The outcome of ex vivo culture for many types of adherent cells is increasingly recognized to depend upon the mechanical properties of the culture substrate. Fibroblast spreading and focal adhesion formation is highly dependent upon the force generated by the fibroblast, as well as the elasticity of the material to which they attach (Balaban et al., 2001, Nat Cell Biol. 3(5):466-72; Choquet et al., 1997, Cell 88(1):39-48). The differentiation of pluripotent mesenchymal stem cells is directly linked to the stiffness of the culture substrate (Engler et al., 2006, Cell. 126(4):677-89). Similarly, expanding myogenic stem cells on soft hydrogel materials leads to enhanced self-renewal and improved engraftment into mice (Gilbert et al., 2010, Science 329(5995):1078-81).

T cells are unlikely to encounter a stimulatory surface with the stiffness of plastic in vivo, and the stiffness of the solid supports used for ex vivo culture of T cells may have important influences on their activation, proliferation, and differentiation that could impact their use in adoptive immunotherapy. It has long been recognized that anti-CDR agonist antibodies such as OKT3 and peptide/MHC complexes require immobilization on solid supports for robust T cell activation (Geppert and Lipsky, 1987, J Immunol. 138(6):1660-6). T cell cytoskeleton integrity and contractility also appear vital for T cell activation (Sims et al., 2007, Cell 129(4):773-85; Ilani et al., 2009, Nat Immunol 10(5):531-9; Valitutti et al., 1995, J Exp Med 181(2):577-84), and models wherein forces applied by the T cell cytoskeleton to ligand-bound TCR modulate and/or trigger TCR/CD3 signaling have been proposed (Sims et al., 2007, Cell 129(4):773-85; Ma et al., 2008, PLoS Biol 6(2):e43). More recent studies have provided direct evidence for force as a mediator of TCR signal transduction (Kim et al., 2009, J Biol Chem 284(45):31028-37; Li et al., 2010, J Immunol. 184(11):5959-63). The demonstration that the immunotyrosine-based activation motif (ITAM) in the CDR chain can be activated by conformational changes in ITAM interaction with the inner leaflet of the plasma membrane provides at least one possible mechanism by which force might be able to mediate signals through the TCR/CD3 complex (Xu et al., 2008, Cell 135(4):702-13). In addition to the direct role of force in TCR/CD3 complex signal transduction, many proteins involved in TCR and co-stimulatory receptor signal transduction directly or indirectly interact with the actin cytoskeleton (Burkhardt et al., 2008, Annu Rev Immunol 26:233-59). The structure and dynamics of the actin cytoskeleton, which is affected by attachment substrate stiffness (Tee et al., 2011, Biophys J 100(5):L25-7), has been reported to play an important role in supporting and regulating signal transduction at the immune synapse (Gomez et al., 2006, Immunity 24(6):741-52; Campi et al., 2005, J Exp Med 202(8): 1031-6).

However, the inadequacy of isolation and expansion methods used for the generation T cell populations has significantly interfered with advances adoptive immunotherapy. Thus, there is a need for methods of producing sufficient numbers of a desired cell population for safe and effective therapeutic use in human patients. There also remains a need for large-scale expansion of T cells for clinical trials including, but not limited to immunotherapy or immunosuppression of cancers, particularly solid tumor cancers.

### SUMMARY OF THE INVENTION

In one aspect, the present invention provides a culture system which includes a biocompatible substrate with tunable rigidity for use in stimulating a T cell. The substrate displays on its surface a first agent that stimulates a TCR/CD3 complex-associated signal in T cells and a second agent that stimulates a CD28 accessory molecule on the surface of T cells. In one embodiment, the first agent is an anti-CD3 antibody. In one embodiment, the second agent is an anti-CD28 antibody. In one embodiment, the system induces the activation, proliferation, and/or differentiation of T cells.

In one embodiment, the substrate also includes a co-stimulatory molecule selected from the group consisting of CD80, CD86, 4-1BBL, OX40L, ICOS-L, ICAM, PD-L1 and PD-L2.

In one embodiment, the substrate is a polymer comprising a synthetic polymer or copolymer prepared from at least one of the group of monomers consisting of acrylic acid, methacrylic acid, ethyleneimine, crotonic acid, acrylamide, ethyl acrylate, methyl methacrylate, 2-hydroxyethyl methacrylate, lactic acid, glycolic acid, .ε-caprolactone, acrolein, cyanoacrylate, bisphenol A, epichlorhydrin, hydroxyalkylacrylates, siloxane, dimethylsiloxane, ethylene oxide, ethylene glycol, hydroxyalkyl-methacrylates, N-substituted acrylamides, N-substituted methacrylamides, N-vinyl-2-pyrrolidone, 2,4-pentadiene-1-ol, vinyl acetate, acrylonitrile, styrene, p-amino-styrene, p-amino-benzyl-styrene, sodium styrene sulfonate, sodium 2-sulfoxyethyl methacrylate, vinyl pyridine, aminoethyl methacrylates, 2-methacryloyloxy-trimethylammonium chloride, N,N′-methylenebisacrylamide-, ethylene glycol dimethacrylates, 2,2′-(p-phenylenedioxy)-diethyl dimethacrylate, divinylbenzene, and triallylamine, methylenebis-(4-phenyl-isocyanate). In one embodiment, the substrate comprises polydimethylsiloxane elastomer (PDMS).

In one embodiment, the substrate exhibits an elastic modulus ranging from about 25 kPa to about 2 MPa. In one embodiment, the substrate is a planar substrate. In another embodiment, the substrate is a spherical substrate. In one embodiment, the substrate is a microbead.

In one aspect, the present invention provides a method of stimulating T cells in culture, where the method includes culturing T cells in the presence of a biocompatible substrate with tunable rigidity. The substrate displays on its surface a first agent that stimulates a TCR/CD3 complex-associated signal in T cells and a second agent that stimulates a CD28 accessory molecule on the surface of T cells. In one embodiment, the first agent is an anti-CD3 antibody. In one embodiment, the second agent is an anti-CD28 antibody.

In one embodiment, the substrate also includes a co-stimulatory molecule selected from the group consisting of CD80, CD86, 4-1BBL, OX40L, ICOS-L, ICAM, PD-L1 and PD-L2.

In one embodiment, the substrate is a polymer comprising a synthetic polymer or copolymer prepared from at least one of the group of monomers consisting of acrylic acid, methacrylic acid, ethyleneimine, crotonic acid, acrylamide, ethyl acrylate, methyl methacrylate, 2-hydroxyethyl methacrylate, lactic acid, glycolic acid, .ε-caprolactone, acrolein, cyanoacrylate, bisphenol A, epichlorhydrin, hydroxyalkylacrylates, siloxane, dimethylsiloxane, ethylene oxide, ethylene glycol, hydroxyalkyl-methacrylates, N-substituted acrylamides, N-substituted methacrylamides, N-vinyl-2-pyrrolidone, 2,4-pentadiene-1-ol, vinyl acetate, acrylonitrile, styrene, p-amino-styrene, p-amino-benzyl-styrene, sodium styrene sulfonate, sodium 2-sulfoxyethyl methacrylate, vinyl pyridine, aminoethyl methacrylates, 2-methacryloyloxy-trimethylammonium chloride, N,N′-methylenebisacrylamide-, ethylene glycol dimethacrylates, 2,2′-(p-phenylenedioxy)-diethyl dimethacrylate, divinylbenzene, and triallylamine, methylenebis-(4-phenyl-isocyanate). In one embodiment, the substrate comprises polydimethylsiloxane elastomer (PDMS).

In one embodiment, the substrate exhibits an elastic modulus ranging from about 25 kPa to about 2 MPa. In one embodiment, the substrate is a planar substrate. In another embodiment, the substrate is a spherical substrate. In one embodiment, the substrate is a microbead.

In one aspect, the present invention provides a culture system which includes a biocompatible substrate with and optimized rigidity for use in stimulating a T cell. The substrate displays on its surface a first agent that stimulates a TCR/CD3 complex-associated signal in T cells and a second agent that stimulates a CD28 accessory molecule on the surface of T cells.

In one aspect, the present invention provides a method of stimulating T cells in culture, where the method includes culturing T cells in the presence of a biocompatible substrate with an optimized rigidity. The substrate displays on its surface a first agent that stimulates a TCR/CD3 complex-associated signal in T cells and a second agent that stimulates a CD28 accessory molecule on the surface of T cells. In one embodiment, the first agent is an anti-CD3 antibody. In one embodiment, the second agent is an anti-CD28 antibody.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1, comprising FIGS. 1A through 1E, is a series of imaged demonstrating that a T cell culture surface exhibiting controlled elastic modulus can be generated using variably cross-linked PDMS.

FIG. 1A is an image depicting the elastic modulus of PDMS substrates. Horizontal bar represents the mean values derived from four independent batches of PDMS.

FIG. 1B is an image demonstrating that PDMS surfaces were coated by incubation with a biotinylated goat-anti-mouse IgG antibody at the indicated concentration. The adsorbed antibody was detected by incubation with horseradish peroxidase conjugated to streptavidin and tetramethylbenzidine, followed by measurement of the optical density (OD) at 450 nm wavelength. The data presented are representative of two independent experiments. Symbols indicate replicate wells performed within the experiment.

FIG. 1C is an image demonstrating that fluorescently-conjugated antibodies against CD3 (OKT3) and CD28 (9.3) were simultaneously applied to PDMS surfaces pre-coated with goat-anti-mouse IgG at 5 mg/mL followed by washing and blocking. The fluorescent signal intensity at the surface for each antibody was measured by fluorescence microscopy, and normalized to the signal intensity observed on the 1:10 PDMS surface. The surfaces were cultured for 2 days in serum-containing culture medium at 37° C., 5% CO2, and the surface fluorescence was measured at the indicated time points. Bars represent mean change in fluorescence intensity with the 95% confidence interval for three independent experiments with five replicates per experiment.

FIG. 1D is an image demonstrating that cellular supernatants were collected from primary human CD4+ T cells grown in complete X-VIVO 15 culture medium on PDMS surfaces coated with OKT3 and clone 9.3 for 24 hrs. IL-2 levels were measured by ELISA. Control cells were stimulated with anti-CD3/anti-CD28-coated microbeads and cultured in wells containing uncoated PDMS as a control. Data were analyzed by a repeated-measures one way ANOVA and a Neuman-Keuls multiple comparison test for posthoc analysis. Values are means+/−SD from four independent experiments (*p<0.05 for PDMS 1-5 versus 1-25; **p<0.05 for PDMS 1-5 versus 1-50 and 1-10 versus 1-50).

FIG. 1E is an image depicting T cells labeled with CFSE that were plated on the indicated PDMS surfaces coated with anti-CD3 and anti-CD28 for 3 days. CFSE content was then assessed by flow cytometry.

FIG. 2, comprising FIGS. 2A through 2D, is a series of imaged demonstrating that human naïve CD4+ T cell proliferation is enhanced by softer surfaces.

FIG. 2A is an image demonstrating that CD45RO-depleted CD4+ T cells were stimulated on PDMS surfaces of varying rigidity in RPMI culture medium for 3 days prior to transfer to uncoated culture vessels. Cell enumeration was performed every other day beginning on day 3 until the number of cells in the culture ceased increasing and the mean cell volume was <350 fL. The maximum number of population doublings is plotted, with the horizontal bars representing the mean and each symbol representing a separate donor. Data were analyzed by a one-way ANOVA (p=0.0261). All groups were compared using a Newman-Keuls multiple comparison test with the difference between the 1:5 and 1:50 group statistically significant as indicated.

FIG. 2B is an image demonstrating that CD45RO-depleted CD4+ T cells were stimulated using OKT3 and clone 9.3 on the PDMS surfaces as indicated for 3 days, and then transferred to uncoated plastic wells. Cells were enumerated at the indicated time points. Data presented is derived from 7 independent experiments with separate donors.

FIG. 2C is an image depicting the mean cell volume of a mixed population of T cells expanded on the PDMS surfaces. Data were analyzed by a one-way ANOVA and a Newman-Keuls multiple comparison test for post-hoc analysis. Values represent means±S.D. from 6 independent healthy donors (*P<0.05; **p<0.05).

FIG. 2D is an image demonstrating CD4+ T cells were stimulated with anti-CD3 and anti-CD28 antibodies, and cultured until the end of log-phase proliferation. The antibodies were immobilized on the PDMS surface or 4.5 μm microbeads as indicated in the figure with each line representing the results from a separate donor. Culture was performed as described for FIG. 2A. Data represent overall number of population doublings achieved following a single round of stimulation. The number of population doublings for cells cultured on soft or stiff PDMS surfaces was compared under each stimulation condition using a paired, two-tailed Student's t-test with the indicated p-value.

FIG. 3, comprising FIGS. 3A through 3C, is a series of images demonstrating that the expansion of mixed human peripheral blood T cells is enhanced on soft substrates.

FIG. 3A is an image depicting representative growth curves for total T cells, CD4+ and CD8+ T cells expanded on 1:5 and 1:50 PDMS coated with OKT3 and clone 9.3 in X-VIVO 15 culture medium. CD4+ and CD8+ T cells were enumerated by flow cytometry.

FIG. 3B is an image depicting the overall number of population doublings achieved for total T cells, CD4+, and CD8+ T cells stimulated by 1:5 or 1:50 PDMS coated with OKT3 and clone 9.3. Results are derived from 6 separate experiments using T cells from independent healthy donors. The 1:5 and 1:50 conditions were compared using a Paired, two-tailed Student's t-test (*P<0.0008 for total T cells grown on a soft surface relative to hard; **P<0.0013 for CD4+ T cells grown on a soft surface relative to hard; ***P<0.0036 for CD8+ T cells grown on a soft surface relative to hard).

FIG. 3C is an image depicting representative data on the expression of the memory markers CD62L and CCR7 in T cell subsets prior to expansion and following expansion on PDMS substrates. The graphs to the right of the flow cytometry plots show a summary of the percent of CD4+ or CD8+ T cells lacking CD62L expression at the end of the culture period. All cultures began with a mixed population of human peripheral blood CD4+ and CD8+ T cells from 7 separate healthy donors.

FIG. 4, comprising FIGS. 4A through 4C, is a series of images depicting that softer substrates increase IL-2 expression independent of mRNA stability.

FIG. 4A is an image demonstrating that cellular supernatants were collected from mixed populations of human T cells grown on PDMS surfaces coated with OKT3 and clone 9.3 for 24 hrs in RPMI culture medium. IL-2 was measured by a human IL-2 specific ELISA. The results shown are derived from 5 separate healthy donors.

FIG. 4B is an image demonstrating that CD4+ T cells were stimulated on PDMS substrates coated with OKT3 and clone 9.3. Cells were harvested after 20-24 hrs and IL-2 mRNA expression was measured by qRT-PCR analysis following normalization to (3-actin. Values represent the means±S.D. from four independent experiments.

FIG. 4C is an image demonstrating that T cells were stimulated on PDMS substrates coated with OKT3 and clone 9.3. After 6 hrs, cyclosporine A was added (t=0 hrs) to the culture to inhibit IL-2 transcription. IL-2 mRNA was measured by qRT-PCR analysis with normalization to the t=6 hrs mRNA expression for each PDMS condition.

FIG. 5, comprising FIGS. 5A through 5C, is a series of imaged demonstrating that stimulatory substrate rigidity influences the frequency of IFN-γ producing cells derived from naïve CD4+ T cell cultures.

FIG. 5A is an image demonstrating that CD45RO-depletion generates CD4+ T cells with a homogenous CD45RA+ naïve phenotype. Data are representative of 3 independent donors. FIG. 5B is an image demonstrating that naïve CD4+ T cells were stimulated on OKT3 and clone 9.3 coated PDMS surfaces in RPMI culture medium and expanded until termination of log phase growth. The T cells were restimulated with PMA and ionomycin for 5 hours in presence of Golgi Stop followed by paraformaldehyde fixation, permeabilization and staining for intracellular IFN-γ. The flow cytometry plots shown are representative of 3 independent donors.

FIG. 5C is an image depicting the mean percentage of IFN-γ positive cells (±SEM) from three independent experiments with separate donors as analyzed in FIG. 5B. The 1:5 and 1:50 conditions were significantly different when compared using a two-tailed Student's t-test (*p=0.0219).

FIG. 6 is a graph illustrating that human T cell expansion is enriched on soft substrates with excel soluble anti-CD3 and anti-CD28. CD4+ T cells were stimulated on PDMS surfaces coated initially with 1 μg/ml goat anti-mouse IgG antibody. Surfaces were blocked as described elsewhere herein. In separate wells, CD4+ T cells were added in combination with soluble OKT3 and clone 9.3 (each at 1 μg/ml) to PDMS surfaces previously coated with 1 μg/ml goat anti mouse IgG. Following three days, the cells were transferred to uncoated culture flasks and expanded until termination of log phase growth. Parallel cultures using standard immobilized OKT3 and 9.3 were performed as a control. Cell number was enumerated by flow cytometry. Data from three independent healthy donors is shown. The 1:5 and 1:50 PDMS conditions for standard coating and soluble OKT3 and clone 9.3 were compared using a paired, one-tailed Student's t-test (P<0.0228 for CD4+ T cells stimulated on standard immobilized antibodies; P<0.0472 for CD4+ T cells stimulated with soluble antibodies).

FIG. 7, comprising FIGS. 7A through 7C, is a series of graphs illustrating that human Treg cell number and function is unchanged following expansion on soft/hard PDMS substrates. T cells were stimulated on PDMS surfaces of varying rigidity coated with antibodies against OKT3 and 9.3. After three days the cells were transferred to uncoated culture flasks and expanded until termination of log phase growth.

FIG. 7A is a graph illustrating the percentage of CD4+ Foxp3+ cells, enumerated by flow cytometry using CD25 (BD Biosciences), CD4, CD127 and FOXP3 (clone PCH101) antibodies from eBioscience Data presented is representative of three independent experiments with separate donors.

FIG. 7B is a graph illustrating the methylation status of the Treg-specific demethylated region (TSDR) in the Foxp3 promoter, assessed following PCR amplification of the FOCP3 TSDR region and sequential restriction enzyme digest of the amplicon to calculate the percentage of methylated CpG islands in the TSDR region of FOXP3. A 0-5% methylation status in the TSDR promoter region was defined as unmethylated. Representative data from two independent donors is shown.

FIG. 7C is a graph illustrating the results of experiments where CD4+ CD25+ and CD4+CD25−T cells expanded from soft/hard surfaces were co-cultured with PBMC's from a separate donor at varying ratios (ranging from 1:1 to 1:4) in the presence of stimulatory CD3ε mAb-coated microbeads for 4-5 days. Proliferation was assessed by labeling the cells with CellTrace (Invitrogen) and analysis by flow cytometry.

FIG. 8, comprising FIGS. 8A through 8D, is as series of graphs illustrating that cytokine expression and exhaustion markers are similar on T cell expanded on soft and stiff PDMS substrates. Total T cells were stimulated on OKT3 and clone 9.3 coated PDMS and expanded until termination of log phase growth. T cells were restimulated with PMA and ionomycin for 5 hrs in the presence of GolgiStop and Brefeldin A followed by surface staining for CD3, CD4, CD8. After paraformaldehyde fixation, the cells permeabilized and stained with monoclonal antibodies to CD27, PD-1, 2B4, Lag-3, IFN-g, TNF-α and Perforin.

FIG. 8A is a series of graphs depicting the proportion of IFN-g and TNF-α expressing cells in the CD4+ subset before and after expansion.

FIG. 8B is a series of graphs depicting the proportion of IFN-g and TNF-α expressing cells in the CD8+ subset before and after expansion.

FIG. 8C is a series of graphs depicting the proportion of Lag3, 2B4 and PD-1 expressing cells in the CD4+ subset before and after expansion.

FIG. 8D is a series of graphs depicting the proportion of Lag3, 2B4 and PD-1 expressing cells in the CD8+ subset before and after expansion.

FIG. 9 is a graph illustrating the results of an experiment demonstrating T cell proliferation, as assessed by carboxyfluoroscein succimidyl ester (CFSE) label dilution to monitor cell division, following 72 hours of activation by PDMS beads of 1:10 Sylgard 184 coated with anti-CD3 and anti-CD28 agonist antibodies, polystyrene beads coated with anti-CD3 and anti-CD28 agonist antibodies, or T cells cultured in the absence of activating antibodies or beads.

FIG. 10 is set of graphs illustrating the results of experiments demonstrating the proliferation of CD4+ T cells over 5 days when cultured with PDMS beads or polystyrene beads coated with anti-CD3 and anti-CD28 agonist antibodies.

FIG. 10A shows population doublings as assessed by enumeration of viable cells in the cultures.

FIG. 10B shows flow cytometry of PDMS bead-activated CD4+ T cells on day 5 of culture. The increase in forward light scatter (a parameter that correlates with cell size) is indicative of the increased metabolic activity associated with activated T cell blasts. The low 7-AAD (Viaprobe) fluorescence demonstrates that the T cell blasts are viable.

### DETAILED DESCRIPTION OF THE INVENTION

The present invention includes compositions and methods for culturing T cells. The T cells are cultured in a culture system comprising a biocompatible substrate wherein the substrate can be manipulated to exhibit different mechanical properties. This is because the present invention is based in part on the discovery that the elastic modulus of a cell culture surface influences the activation, proliferation, and differentiation of T cells cultured therewith. Accordingly, the invention provides a cell culture system have a tunable rigidity for the culturing of T cells.

In one aspect of the present invention, T cell expansion can be performed by isolating T cells from a desired cell source and subsequently culture expanding the cells in the presence of a primary signal and a co-stimulatory signal in combination with the biocompatible substance with a tunable rigidity. Agents useful for stimulating a primary signal and an a co-stimulatory signal on T cells may be used in soluble form, attached to the surface of a cell, or immobilized on the surface of the tunable rigidity biocompatible substance.

In a preferred embodiment both primary and co-stimulatory agents are co-immobilized on the surface of the biocompatible substrate. In one embodiment, the molecule providing the primary activation signal, such as a CD3 ligand, and the co-stimulatory molecule, such as a CD28 ligand are coupled to or loaded on the same surface, for example, the biocompatible substrate. Preferably, the substrate is a material with a bulk modulus that can be controlled by changing the ratio of base elastomer to curing agent.

In other embodiments, at least a portion of the active cell population is stored for later implantation/infusion. The population may be divided into more than one aliquot or unit such that part of the population of T cells is retained for later application while part is applied immediately to the patient. Moderate to long-term storage of all or part of the cells in a cell bank is also within the scope of this invention.

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

The term “antibody,” as used herein, refers to an immunoglobulin molecule which specifically binds with an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab)₂, as well as single chain antibodies and humanized antibodies (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426).

The term “agent,” “ligand,” or “agent that binds a cell surface moiety,” as used herein, refers to a molecule that binds to a defined population of cells. The agent may bind any cell surface moiety, such as a receptor, an antigenic determinant, or other binding site present on the target cell population. The agent may be a protein, peptide, antibody and antibody fragments thereof, fusion proteins, synthetic molecule, an organic molecule (e.g., a small molecule), a carbohydrate, or the like. Within the specification and in the context of T cell stimulation, antibodies and natural ligands are used as prototypical examples of such agents.

The terms “agent that binds a cell surface moiety” and “cell surface moiety”, as used herein, are used in the context of a ligand/anti-ligand pair. Accordingly, these molecules should be viewed as a complementary/anti-complementary set of molecules that demonstrate specific binding, generally of relatively high affinity.

As used herein, the term “autologous” is meant to refer to any material derived from the same individual to which it is later to be re-introduced into the individual.

“Allogeneic” refers to a graft derived from a different animal of the same species.

“Xenogeneic” refers to a graft derived from an animal of a different species.

The term “T-cell” as used herein is defined as a thymus-derived cell that participates in a variety of cell-mediated immune reactions.

The term “B-cell” as used herein is defined as a cell derived from the bone marrow and/or spleen. B cells can develop into plasma cells which produce antibodies.

“Effective amount” or “therapeutically effective amount” are used interchangeably herein, and refer to an amount of a compound, formulation, material, or composition, as described herein effective to achieve a particular biological result. Such results may include, but are not limited to, the inhibition of virus infection as determined by any means suitable in the art.

As used herein “endogenous” refers to any material from or produced inside an organism, cell, tissue or system.

As used herein, the term “exogenous” refers to any material introduced from or produced outside an organism, cell, tissue or system.

As used herein, an “instructional material” includes a publication, a recording, a diagram, or any other medium of expression which can be used to communicate the usefulness of the compositions and methods of the invention. The instructional material of the kit of the invention may, for example, be affixed to a container which contains the nucleic acid, peptide, and/or composition of the invention or be shipped together with a container which contains the nucleic acid, peptide, and/or composition. Alternatively, the instructional material may be shipped separately from the container with the intention that the instructional material and the compound be used cooperatively by the recipient.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

An “isolated nucleic acid” refers to a nucleic acid segment or fragment which has been separated from sequences which flank it in a naturally occurring state, i.e., a DNA fragment which has been removed from the sequences which are normally adjacent to the fragment, i.e., the sequences adjacent to the fragment in a genome in which it naturally occurs. The term also applies to nucleic acids which have been substantially purified from other components which naturally accompany the nucleic acid, i.e., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (i.e., as a cDNA or a genomic or cDNA fragment produced by PCR or restriction enzyme digestion) independent of other sequences. It also includes a recombinant DNA which is part of a hybrid gene encoding additional polypeptide sequence.

As used herein, the term “modulate” is meant to refer to any change in biological state, i.e. increasing, decreasing, and the like.

“Quiescent,” as used herein, refers to a cell state wherein the cell is not actively proliferating.

The term “subject” is intended to include living organisms in which an immune response can be elicited (e.g., mammals).

As used herein, a “substantially purified” cell is a cell that is essentially free of other cell types. A substantially purified cell also refers to a cell which has been separated from other cell types with which it is normally associated in its naturally occurring state. In some instances, a population of substantially purified cells refers to a homogenous population of cells. In other instances, this term refers simply to cell that have been separated from the cells with which they are naturally associated in their natural state. In some embodiments, the cells are cultured in vitro. In other embodiments, the cells are not cultured in vitro.

The term “therapeutic” as used herein means a treatment and/or prophylaxis. A therapeutic effect is obtained by suppression, remission, or eradication of a disease state.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

By the term “stimulation,” is meant a primary response induced by binding of a stimulatory molecule (e.g., a TCR/CD3 complex) with its cognate ligand thereby mediating a signal transduction event, such as, but not limited to, signal transduction via the TCR/CD3 complex. Stimulation can mediate altered expression of certain molecules, such as downregulation of TGF-β, and/or reorganization of cytoskeletal structures, and the like.

“Activation,” as used herein, refers to the state of a T cell that has been sufficiently stimulated to induce detectable cellular proliferation. Activation can also be associated with induced cytokine production, and detectable effector functions. The term “activated T cells” refers to, among other things, T cells that are undergoing cell division.

By the term “specifically binds,” as used herein, is meant an antibody, or a ligand, which recognizes and binds with a cognate binding partner (e.g., a stimulatory and/or costimulatory molecule present on a T cell) protein present in a sample, but which antibody or ligand does not substantially recognize or bind other molecules in the sample.

A “stimulatory ligand,” as used herein, means a ligand that when present on an antigen presenting cell (e.g., an aAPC, a dendritic cell, a B-cell, and the like) can specifically bind with a cognate binding partner (referred to herein as a “stimulatory molecule”) on a T cell, thereby mediating a primary response by the T cell, including, but not limited to, activation, initiation of an immune response, proliferation, and the like. Stimulatory ligands are well-known in the art and encompass, inter alia, an MHC Class I molecule loaded with a peptide, an anti-CD3 antibody, a superagonist anti-CD28 antibody, and a superagonist anti-CD2 antibody.

A “stimulatory molecule,” as the term is used herein, means a molecule on a T cell that specifically binds with a cognate stimulatory ligand present on an antigen presenting cell (e.g., an aAPC of the invention, among others).

A “co-stimulatory signal,” as used herein, refers to a signal, which in combination with a primary signal, such as TCR/CD3 ligation, leads to T cell proliferation and/or upregulation or downregulation of key molecules.

A “co-stimulatory molecule” refers to the cognate binding partner on a T cell that specifically binds with a co-stimulatory ligand, thereby mediating a co-stimulatory response by the T cell, such as, but not limited to, proliferation. Co-stimulatory molecules include, but are not limited to an MHC class I molecule, BTLA and a Toll ligand receptor.

“Co-stimulatory ligand,” as the term is used herein, includes a molecule on an antigen presenting cell (e.g., an aAPC, dendritic cell, B cell, and the like) that specifically binds a cognate co-stimulatory molecule on a T cell, thereby providing a signal which, in addition to the primary signal provided by, for instance, binding of a TCR/CD3 complex with an MHC molecule loaded with peptide, mediates a T cell response, including, but not limited to, proliferation, activation, differentiation, and the like. A co-stimulatory ligand can include, but is not limited to, CD7, B7-1 (CD80), B7-2 (CD86), PD-L1, PD-L2, 4-1BBL, OX40L, inducible costimulatory ligand (ICOS-L), intercellular adhesion molecule (ICAM), CD30L, CD40, CD70, CD83, HLA-G, MICA, MICB, HVEM, lymphotoxin beta receptor, 3/TR6, ILT3, ILT4, HVEM, an agonist or antibody that binds Toll ligand receptor and a ligand that specifically binds with B7-H3. A co-stimulatory ligand also encompasses, inter alia, an antibody that specifically binds with a co-stimulatory molecule present on a T cell, such as, but not limited to, CD27, CD28, 4-1BB, OX40, CD30, CD40, PD-1, ICOS, lymphocyte function-associated antigen-1 (LFA-1), CD2, CD7, LIGHT, NKG2C, B7-H3, and a ligand that specifically binds with CD83.

### Description

The present invention provides a system and method for culturing and expanding T cells. The method includes expanding T cells using a biocompatible polymer substrate whereby the activation and expansion of the cells can be regulated by manipulating the stiffness of the substrate. Accordingly, the present invention allows for expansion of any T cell population and substantially increasing the number of T cells for subsequent use following expansion.

In one embodiment, the rigidity of the substrate can be manipulated to effect T cell differentiation.

Regardless of whether the T cells are being expanded or differentiated, the biocompatible polymer substrate is used for immobilizing T cell ligands. The ligands for immobilization on the substrate vary based on the particular desired biological activity exhibited by the culture system.

The present invention provides compositions and methods for their use to expand a T cells as well as numerous therapeutic uses relating to expansion, stimulation and differentiation of T cells.

### Tunable Rigidity

The present invention provides a method for regulating the expansion and differentiation of T cells on a biocompatible substrate whereby the rigidity of the substrate can be manipulated. In some aspects of the invention, cell expansion and differentiation is influenced by stiffness (elasticity) of the substrate.

Regardless of geometry, the intrinsic resistance of a solid to a stress is measured by the solid's elastic (or Young's) modulus E, which is most simply obtained by applying a force, such as hanging a weight, to a section of tissue or other material and then measuring the relative change in length or strain. Another common method to obtain E involves controlled macro- or micro-indentation, including atomic force microscopy (AFM). The elastic modulus E is discussed, e.g., by Sugawara et al., Hearing Research 192:57-64 (2004); Taylor et al. J. Biomech. 37:1263-1269 (2004).

The present invention is based on the observation that T cell activating and differentiation agents can be immobilized on a biocompatible substrate that is tunable with respect to rigidity whereby the rigidity has an influence on the culturing of the cells. Using these tunable systems, the elastic modulus of a cell culture surface of the invention can influence the activation, proliferation, and differentiation of T cells in ways that could be advantageous for adoptive immunotherapy.

The rigidity of the biocompatible surface can be manipulated where the Young's modulus E can be well-controlled by varying the concentration of cross-linker. In one embodiment, the material of the biocompatible surface represents a material with bulk modulus that can be controlled by changing the ratio of base elastomer to curing agent. For example, elastic moduli ranging from 5 kPa to 10 MPa, preferably 25 kPa (1:50 base:cure) to 2 MPa (1:5 base:cure) is desired. Substrate surfaces can be prepared by mixing the appropriate ratio of base polymer to curing agent to achieve the desired ranges of rigidity.

The biocompatible substrate of the present invention is not limited by any particular geometry. For example, in one embodiment, the biocompatible substrate is planar. In another embodiment, the biocompatible substrate is spherical. For example, in an embodiment, the biocompatible substrate is a microbead. In one embodiment, the substrate has a diameter of 1-1000 μm. In another embodiment, the substrate has a diameter of 5-500 μm. In another embodiment, the substrate has a diameter of 10-100 μm. In another embodiment, the substrate has a diameter of 20-50 μm. In another embodiment, the substrate has a diameter of less than 1 μm. For example, in one embodiment, the substrate has a diameter of 1-1000 nm.

A variety of polymers from synthetic and/or natural sources can be used to compose the biocompatible substrate of the invention. A tunable substrate can be made from a polymer comprising one monomer or subunit or from a polymer comprising a plurality of monomers or subunits. For example, lactic or polylactic acid or glycolic or polyglycolic acid can be utilized to form poly(lactide) (PLA) or poly(L-lactide) (PLLA) substrates or poly(glycolide) (PGA) substrates. Substrates of the invention can also be made from polymers comprising more than one monomer or subunit thus forming a copolymer, terpolymer, etc. For example, lactic or polylactic acid and be combined with glycolic acid or polyglycolic acid to form the copolymer poly(lactide-co-glycolide) (PLGA). Other copolymers of use in the present disclosure include poly(ethylene-co-vinyl) alcohol.

The polymer(s) may be natural polymers, biological polymers, synthetic polymers, or a combination thereof. In various embodiments, the polymer fibers used to create the substrate scaffolds of the present invention are selected from aliphatic polyesters, polyhydroxyalkanoates, polyurethanes, polyalkylene oxides, polydimethylsiloxane, polyvinylalcohol, polyvinylpyrrolidone, polylysine, collagen, gelatin, laminin, fibronectin, elastin, alginate, fibrin, hyaluronic acid, proteoglycans, polypeptides, polysaccharides and combinations thereof.

In some embodiments, a substrate of the invention comprises one or more of a member selected from aliphatic polyesters, polyhydroxyalkanoates, polyurethanes, polyalkylene oxides, polydimethylsiloxane, polyvinylalcohol, polyvinylpyrrolidone, polylysine, collagen, gelatin, laminin, fibronectin, elastin, alginate, fibrin, hyaluronic acid, proteoglycans, polypeptides, polysaccharides and combinations thereof.

In one embodiment, the aliphatic polyester can be linear or branched. In some embodiments, the aliphatic polyester is linear and is selected from D-lactic acid, L-lactic acid, lactide, poly(lactic acid), poly(lactide) glycolic acid, poly(glycolic acid), poly(glycolide), glycolide, poly(lactide-co-glycolide), poly(lactic acid-co-glycolic acid), polycaprolactone and combinations thereof. In some embodiments, the aliphatic polyester is branched and is selected from D-lactic acid, L-lactic acid, lactide, poly(lactic acid), poly(lactide) glycolic acid, poly(glycolic acid), poly(glycolide), glycolide, poly(lactide-co-glycolide), poly(lactic acid-co-glycolic acid), polycaprolactone and combinations thereof. In some embodiments, the aliphatic polyester is conjugated to a linker or a biomolecule.

In some embodiments, the polyalkylene oxide is selected from polyethylene oxide, polyethylene glycol, polypropylene oxide, polypropylene glycol and combinations thereof.

In some embodiments, the polymer substance scaffolds of the invention are biodegradable. In some embodiments, the substance of the invention comprises biodegradable polymers. In some embodiments, the biodegradable polymers comprise a monomer which is a member selected from lactic acid and glycolic acid. In another exemplary embodiment, the biodegradable polymers are poly(lactic acid), poly(glycolic acid) or a copolymer thereof. In some embodiments, the biodegradable polymers are those which are approved by the FDA for clinical use, such as poly(lactic acid) and poly(glycolic acid).

In various aspects, substances comprising biodegradable polymers can be used to guide the morphogenesis of tissue and/or to gradually degrade after the assembly of the tissue. The degradation rate of the polymers can be tailored by one of skill in the art to match the tissue generation rate. For example, if a polymer that biodegrades quickly is desired, a combination of approximately 50:50 polylactic acid to glycolic acid or polyglycolic acid can be selected to form the copolymer poly(lactide-co-glycolide). Additional ways to increase polymer biodegradability can involve selecting a hydrophilic copolymer (for example, polyethylene glycol), decreasing the molecular weight of the polymer, as higher molecular weight often means a slower degradation rate, and decreasing the density in the scaffolds, as lower density can lead to more water absorption and faster degradation.

In various embodiments, the polymer content of the scaffolds is adjusted to match the architecture and desired physical properties. In some embodiments, the polymer content of the scaffolds can be adjusted to closely match an elastic modulus of about 5 kPa to 5 MPa, preferably 25 kPa to 2 MPa.

The tunable rigidity culture system of the present invention can be used with any prior art systems and methods for culturing cells. The tunable rigidity culture system of the invention is based on the discovery of a previously unrecognized parameter for controlling T cell activation, expansion and differentiation. This novel parameter is the elasticity of a substrate whereby controlling the elasticity allows for controlling T cell activation, expansion and differentiation. For example, softer substrates comprising stimulatory agents for inducing proliferation of T cells yields a larger number of expanded T cells compared to a more rigid substrate. Therefore, the softer substrates of the invention provide an improvement over current hard plastic based culture systems.

### Sources of T Cells

Prior to expansion, a source of T cells is obtained from a subject. The term “subject” is intended to include living organisms in which an immune response can be elicited (e.g., mammals). Examples of subjects include humans, dogs, cats, mice, rats, and transgenic species thereof. T cells can be obtained from a number of sources, including peripheral blood mononuclear cells, bone marrow, lymph node tissue, cord blood, thymus tissue, tissue from a site of infection, ascites, pleural effusion, spleen tissue, and tumors. In certain embodiments of the present invention, any number of T cell lines available in the art, may be used. In certain embodiments of the present invention, T cells can be obtained from a unit of blood collected from a subject using any number of techniques known to the skilled artisan, such as ficoll separation. In one preferred embodiment, cells from the circulating blood of an individual are obtained by apheresis or leukapheresis. The apheresis product typically contains lymphocytes, including T cells, monocytes, granulocytes, B cells, other nucleated white blood cells, red blood cells, and platelets. In one embodiment, the cells collected by apheresis may be washed to remove the plasma fraction and to place the cells in an appropriate buffer or media for subsequent processing steps. In one embodiment of the invention, the cells are washed with phosphate buffered saline (PBS). In an alternative embodiment, the wash solution lacks calcium and may lack magnesium or may lack many if not all divalent cations. Again, surprisingly, initial activation steps in the absence of calcium lead to magnified activation. As those of ordinary skill in the art would readily appreciate a washing step may be accomplished by methods known to those in the art, such as by using a semi-automated “flow-through” centrifuge (for example, the Cobe 2991 cell processor, the Baxter CytoMate, or the Haemonetics Cell Saver 5) according to the manufacturer's instructions. After washing, the cells may be resuspended in a variety of biocompatible buffers, such as, for example, Ca-free, Mg-free PBS, PlasmaLyte A, or other saline solution with or without buffer. Alternatively, the undesirable components of the apheresis sample may be removed and the cells directly resuspended in culture media.

Enrichment of a T cell population by negative selection can be accomplished with a combination of antibodies directed to surface markers unique to the negatively selected cells. One method is cell sorting and/or selection via negative magnetic immunoadherence or flow cytometry that uses a cocktail of monoclonal antibodies directed to cell surface markers present on the cells negatively selected. For example, to enrich for CD4⁺ cells by negative selection, a monoclonal antibody cocktail typically includes antibodies to CD14, CD20, CD11b, CD16, HLA-DR, and CD8. In certain embodiments, it may be desirable to enrich for or positively select for regulatory T cells which typically express CD4⁺, CD25⁺, CD62L^{hi}, GITR⁺, and FoxP3⁺. Alternatively, in certain embodiments, T regulatory cells are depleted by anti-C25 conjugated beads or other similar method of selection.

For isolation of a desired population of cells by positive or negative selection, the concentration of cells and surface (e.g., particles such as beads) can be varied. In certain embodiments, it may be desirable to significantly decrease the volume in which beads and cells are mixed together (i.e., increase the concentration of cells), to ensure maximum contact of cells and beads. For example, in one embodiment, a concentration of 2 billion cells/ml is used. In one embodiment, a concentration of 1 billion cells/ml is used. In a further embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. Using high concentrations can result in increased cell yield, cell activation, and cell expansion. Further, use of high cell concentrations allows more efficient capture of cells that may weakly express target antigens of interest, such as CD28-negative T cells, or from samples where there are many tumor cells present (i.e., leukemic blood, tumor tissue, etc.). Such populations of cells may have therapeutic value and would be desirable to obtain. For example, using high concentration of cells allows more efficient selection of CD8⁺ T cells that normally have weaker CD28 expression.

If desired or necessary, monocyte populations (i.e., CD14⁺ cells) may be depleted from blood preparations prior to ex vivo expansion by a variety of methodologies, including anti-CD14 coated beads or columns, or utilization of the phagocytotic activity of these cells to facilitate removal, or by the use of counterflow centrifugal elutriation. Accordingly, in one embodiment, the invention uses paramagnetic particles of a size sufficient to be engulfed by phagocytotic monocytes. In certain embodiments, the paramagnetic particles are commercially available beads, for example, those produced by Dynal AS under the trade name Dynabeads™. Exemplary Dynabeads™ in this regard are M-280, M-450, and M-500. In one aspect, other non-specific cells are removed by coating the paramagnetic particles with “irrelevant” proteins (e.g., serum proteins or antibodies). Irrelevant proteins and antibodies include those proteins and antibodies or fragments thereof that do not specifically target the T cells to be expanded. In certain embodiments the irrelevant beads include beads coated with sheep anti-mouse antibodies, goat anti-mouse antibodies, and human serum albumin.

In a further embodiment of the present invention, T cells are obtained from a patient directly following treatment. In this regard, it has been observed that following certain cancer treatments, in particular treatments with drugs that damage the immune system, shortly after treatment during the period when patients would normally be recovering from the treatment, the quality of T cells obtained may be optimal or improved for their ability to expand ex vivo. Likewise, following ex vivo manipulation using the methods described herein, these cells may be in a preferred state for enhanced engraftment and in vivo expansion. Thus, it is contemplated within the context of the present invention to collect blood cells, including T cells, dendritic cells, or other cells of the hematopoetic lineage, during this recovery phase. Further, in certain embodiments, mobilization (for example, mobilization with GM-CSF) and conditioning regimens can be used to create a condition in a subject wherein repopulation, recirculation, regeneration, and/or expansion of particular cell types is favored, especially during a defined window of time following therapy. Illustrative cell types include T cells, B cells, dendritic cells, and other cells of the immune system.

### Culture System

As indicated above, the methods of the present invention preferably use agents/ligands bound to a surface to culture T cells. The surface may be any surface capable of having an agent/ligand bound thereto or integrated into and that is biocompatible, that is, substantially non-toxic to the target cells to be stimulated. The biocompatible surface may be biodegradable or non-biodegradable. The surface may be natural or synthetic, and a synthetic surface may be a polymer. The surface of the biocompatible substance of the invention represents a material with a bulk modulus that can be controlled by changing the ratio of base elastomer to curing agent.

An agent may be attached or coupled to, or integrated into a surface by a variety of methods known and available in the art. The agent may be a natural ligand, a protein ligand, or a synthetic ligand. The attachment may be covalent or noncovalent, electrostatic, or hydrophobic and may be accomplished by a variety of attachment means, including for example, chemical, mechanical, enzymatic, electrostatic, or other means whereby a ligand is capable of stimulating the cells. For example, the antibody to a ligand first may be attached to a surface, or avidin or streptavidin may be attached to the surface for binding to a biotinylated ligand. The antibody to the ligand may be attached to the surface via an anti-idiotype antibody. Another example includes using protein A or protein G, or other non-specific antibody binding molecules, attached to surfaces to bind an antibody. Alternatively, the ligand may be attached to the surface by chemical means, such as cross-linking to the surface, using commercially available cross-linking reagents (Pierce, Rockford, Ill.) or other means. In certain embodiments, the ligands are covalently bound to the surface.

In one aspect, the agent, such as certain ligands may be of singular origin or multiple origins and may be antibodies or fragments thereof while in another aspect, when utilizing T cells, the co-stimulatory ligand is a B7 molecule (e.g., B7-1, B7-2). These ligands are coupled to the surface by any of the different attachment means discussed above. The B7 molecule to be coupled to the surface may be isolated from a cell expressing the co-stimulatory molecule, or obtained using standard recombinant DNA technology and expression systems that allow for production and isolation of the co-stimulatory molecule(s) as described herein. Fragments, mutants, or variants of a B7 molecule that retain the capability to trigger a co-stimulatory signal in T cells when coupled to the surface of a cell can also be used. Furthermore, one of ordinary skill in the art will recognize that any ligand useful in the activation and induction of proliferation of a subset of T cells may also be immobilized on the surface of the biocompatible substance of the invention. In addition, while covalent binding of the ligand to the surface is one preferred methodology, adsorption or capture by a secondary monoclonal antibody may also be used. The amount of a particular ligand attached to a surface may be readily determined by flow cytometric analysis if the surface is that of beads or determined by enzyme-linked immunosorbent assay (ELISA) if the surface is a tissue culture dish, mesh, fibers, bags, for example.

In a particular embodiment, the stimulatory form of a B7 molecule or an anti-CD28 antibody or fragment thereof is attached to the same solid phase surface as the agent that stimulates the TCR/CD3 complex, such as an anti-CD3 antibody. In an additional embodiment, the stimulatory form of a 4-1BB molecule or an anti-4-1BB antibody or fragment thereof is attached to the same solid phase surface as the agent that stimulates the TCR/CD3 complex, such as an anti-CD3 antibody. In addition to anti-CD3 antibodies, other antibodies that bind to receptors that mimic antigen signals may be used. For example, surfaces of the invention may be coated with combinations of anti-CD2 antibodies and a B7 molecule and in particular anti-CD3 antibodies and anti-CD28 antibodies. In further embodiments, the surfaces may be coated with three or more agents, such as combinations of any of the agents described herein, for example, anti-CD3 antibodies, anti-CD28 antibodies, and anti-4-1BB antibodies.

When coupled to a surface, the agents may be coupled to the same surface (i.e., in “cis” formation) or to separate surfaces (i.e., in “trans” formation). Alternatively, one agent may be coupled to a surface and the other agent in solution. In one embodiment, the agent providing the co-stimulatory signal is bound to a cell surface and the agent providing the primary activation signal is in solution or coupled to a surface. In a preferred embodiment, the two agents are immobilized on beads, either on the same bead, i.e., “cis,” or to separate beads, i.e., “trans.” By way of example, the agent providing the primary activation signal is an anti-CD3 antibody and the agent providing the co-stimulatory signal is an anti-CD28 antibody; and both agents are co-immobilized to the same bead in equivalent molecular amounts. In one embodiment, a 1:1 ratio of each antibody bound to the beads for CD4⁺ T cell expansion and T cell growth is used. In certain aspects of the present invention, a ratio of anti CD3:CD28 antibodies bound to the beads is used such that an increase in T cell expansion is observed as compared to the expansion observed using a ratio of 1:1. In one particular embodiment an increase of from about 0.5 to about 3 fold is observed as compared to the expansion observed using a ratio of 1:1. In one embodiment, the ratio of CD3:CD28 antibody bound to the beads ranges from 100:1 to 1:100 and all integer values there between. In one aspect of the present invention, more anti-CD28 antibody is bound to the particles than anti-CD3 antibody, i.e. the ratio of CD3:CD28 is less than one. In certain embodiments of the invention, the ratio of anti CD28 antibody to anti CD3 antibody bound to the beads is greater than 2:1. In one particular embodiment, a 1:100 CD3:CD28 ratio of antibody bound to beads is used. In another embodiment, a 1:75 CD3:CD28 ratio of antibody bound to beads is used. In a further embodiment, a 1:50 CD3:CD28 ratio of antibody bound to beads is used. In another embodiment, a 1:30 CD3:CD28 ratio of antibody bound to beads is used. In one preferred embodiment, a 1:10 CD3:CD28 ratio of antibody bound to beads is used. In another embodiment, a 1:3 CD3:CD28 ratio of antibody bound to the beads is used. In yet another embodiment, a 3:1 CD3:CD28 ratio of antibody bound to the beads is used.

### Expansion of T Cells

The present invention further comprises a method of multiplying, expanding or otherwise culturing a T cell isolated using the methods disclosed herein or methods generally known in the art. As demonstrated by the data disclosed herein, multiplying a T cell isolated by the methods of the present invention can by multiplied by about 10 fold, 100 fold, 1000 fold, or more using the methods disclosed herein. Following isolation, a T cell is incubated in cell medium in a culture apparatus for a period of time or until the cells reach confluency before passing the cells to another culture apparatus. The culturing apparatus can be of any culture apparatus commonly used for culturing cells in vitro. Preferably, the level of confluence is greater than 70% before passing the cells to another culture apparatus. More preferably, the level of confluence is greater than 90%. A period of time can be any time suitable for the culture of cells in vitro. T cell medium may be replaced during the culture of the T cells at any time. Preferably, the T cell medium is replaced about every 2 to 3 days. T cells are then harvested from the culture apparatus whereupon the T cells can be used immediately or cryopreserved to be stored for use at a later time. T cells may be harvested by trypsinization, EDTA treatment, or any other procedure used to harvest cells from a culture apparatus.

In one embodiment of the invention, the T cells may be stimulated by a single agent. In another embodiment, T cells are stimulated with two agents, one that induces a primary signal and a second that is a co-stimulatory signal. Ligands useful for stimulating a single signal or stimulating a primary signal and an accessory molecule that stimulates a second signal may be used in soluble form, attached to the surface of a cell or immobilized on a surface as described herein. In a preferred embodiment both primary and secondary agents are co-immobilized on a surface, for example a bead or an artificial presenting cell (aAPC). In one embodiment, the molecule providing the primary activation signal, such as a CD3 ligand, and the co-stimulatory molecule, such as a CD28 ligand are coupled to or loaded on the same surface, for example, a particle or an aAPC.

Various terms are used to describe cells in culture. Cell culture refers generally to cells taken from a living organism and grown under controlled condition. A primary cell culture is a culture of cells, tissues or organs taken directly from an organism and before the first subculture. Cells are expanded in culture when they are placed in a growth medium under conditions that facilitate cell growth and/or division, resulting in a larger population of the cells. When cells are expanded in culture, the rate of cell proliferation is typically measured by the amount of time required for the cells to double in number, otherwise known as the doubling time.

Each round of subculturing is referred to as a passage. When cells are subcultured, they are referred to as having been passaged. A specific population of cells, or a cell line, is sometimes referred to or characterized by the number of times it has been passaged. For example, a cultured cell population that has been passaged ten times may be referred to as a P10 culture. The primary culture, i.e., the first culture following the isolation of cells from tissue, is designated P0. Following the first subculture, the cells are described as a secondary culture (P1 or passage 1). After the second subculture, the cells become a tertiary culture (P2 or passage 2), and so on. It will be understood by those of skill in the art that there may be many population doublings during the period of passaging; therefore the number of population doublings of a culture is greater than the passage number. The expansion of cells (i.e., the number of population doublings) during the period between passaging depends on many factors, including but is not limited to the seeding density, substrate, medium, and time between passaging.

The medium used to multiply the T cells of the present invention comprises an agent that can stimulate CD3 and CD28 on the T cell. For example, an agent that can stimulate CD3 is an antibody to CD3, and an agent that can stimulate CD28 is an antibody to CD28. Preferably, the agent is immobilized on a biocompatible tunable substrate. This is because, as demonstrated by the data disclosed herein, and increase in overall culture yield is observed when comparing the softest substrate (1:50 cross-linker to base ratio) to the stiffest substrate (1:5 cross-linker to base ratio) tested.

### Methods of Use and Pharmaceutical Compositions

Generally, the cells activated and expanded as described herein may be utilized in the treatment and prevention of various diseases. The present invention provides a platform for the ex vivo culture of T cells for adoptive immunotherapy with potential advantages over currently used rigid plastic surfaces.

Pharmaceutical compositions of the present invention may be administered in a manner appropriate to the disease to be treated (or prevented). The quantity and frequency of administration will be determined by such factors as the condition of the patient, and the type and severity of the patient's disease, although appropriate dosages may be determined by clinical trials.

The immune response induced in a subject by administering T cells activated and expanded using the methods described herein, or other methods known in the art wherein T cells are stimulated and expanded to therapeutic levels, may include cellular immune responses mediated by cytotoxic T cells, capable of killing tumor and infected cells, regulatory T cells, and helper T cell responses. Humoral immune responses, mediated primarily by helper T cells capable of activating B cells thus leading to antibody production, may also be induced. A variety of techniques may be used for analyzing the type of immune responses induced by the compositions of the present invention, which are well described in the art; e.g., Coligan et al. Current Protocols in Immunology, John Wiley & Sons Inc. (1994).

When “an immunologically effective amount”, “an anti-tumor effective amount”, “an tumor-inhibiting effective amount”, or “therapeutic amount” is indicated, the precise amount of the compositions of the present invention to be administered can be determined by a physician with consideration of individual differences in age, weight, tumor size, extent of infection or metastasis, and condition of the patient (subject). It can generally be stated that a pharmaceutical composition comprising the T cells described herein may be administered at a dosage of 10⁴ to 10⁹ cells/kg body weight, preferably 10⁵ to 10⁶ cells/kg body weight, including all integer values within those ranges. T cell compositions may also be administered multiple times at these dosages. The cells can be administered by using infusion techniques that are commonly known in immunotherapy (see, e.g., Rosenberg et al., New Eng. J. of Med. 319:1676, 1988). The optimal dosage and treatment regime for a particular patient can readily be determined by one skilled in the art of medicine by monitoring the patient for signs of disease and adjusting the treatment accordingly.

Typically, in adoptive immunotherapy studies, antigen-specific T cells are administered approximately at 2×10⁹ to 2×10¹¹ cells to the patient. (See, e.g., U.S. Pat. No. 5,057,423). In some aspects of the present invention, particularly in the use of allogeneic or xenogeneic cells, lower numbers of cells, in the range of 10⁶/kilogram (10⁶-10¹¹ per patient) may be administered. In certain embodiments, T cells are administered at 1×10⁵, 1×10⁶, 1×10⁷, 1×10⁸, 2×10⁸, 2×10⁹, 1×10¹⁰, 2×10¹⁰, 1×10¹¹, 5×10¹¹, or 1×10¹² cells to the subject. T cell compositions may be administered multiple times at dosages within these ranges. The cells may be autologous or heterologous to the patient undergoing therapy. If desired, the treatment may also include administration of mitogens (e.g., PHA) or lymphokines, cytokines, and/or chemokines (e.g., GM-CSF, IL-4, IL-7, IL-13, FIt3-L, RANTES, MIP1α, etc.) as described herein to enhance induction of the immune response.

In certain embodiments, it may be desired to administer activated T cells to a subject and then subsequently redraw blood (or have a leukapheresis performed), activate T cells therefrom according to the present invention, and reinfuse the patient with these activated and expanded T cells. This process can be carried out multiple times every few weeks. In certain embodiments, T cells can be activated from blood draws of from 10 cc to 400 cc. In certain embodiments, T cells are activated from blood draws of 20 cc, 30 cc, 40 cc, 50 cc, 60 cc, 70 cc, 80 cc, 90 cc, or 100 cc. Not to be bound by theory, using this multiple blood draw/multiple reinfusion protocol, may select out certain populations of T cells.

The administration of the subject compositions may be carried out in any convenient manner, including by aerosol inhalation, injection, ingestion, transfusion, implantation or transplantation. The compositions described herein may be administered to a patient subcutaneously, intradermally, intratumorally, intranodally, intramedullary, intramuscularly, by intravenous (i.v.) injection, or intraperitoneally. In one embodiment, the T cell compositions of the present invention are administered to a patient by intradermal or subcutaneous injection. In another embodiment, the T cell compositions of the present invention are preferably administered by i.v. injection. The compositions of T cells may be injected directly into a tumor, lymph node, or site of infection.

In yet another embodiment, the pharmaceutical composition can be delivered in a controlled release system. In one embodiment, a pump may be used (see Langer, 1990, Science 249:1527-1533; Sefton 1987, CRC Crit. Ref. Biomed. Eng. 14:201; Buchwald et al., 1980; Surgery 88:507; Saudek et al., 1989, N. Engl. J. Med. 321:574). In another embodiment, polymeric materials can be used (see Medical Applications of Controlled Release, 1974, Langer and Wise (eds.), CRC Pres., Boca Raton, Fla.; Controlled Drug Bioavailability, Drug Product Design and Performance, 1984, Smolen and Ball (eds.), Wiley, New York; Ranger and Peppas, 1983; J. Macromol. Sci. Rev. Macromol. Chem. 23:61; see also Levy et al., 1985, Science 228:190; During et al., 1989, Ann. Neurol. 25:351; Howard et al., 1989, J. Neurosurg. 71:105). In yet another embodiment, a controlled release system can be placed in proximity of the therapeutic target, thus requiring only a fraction of the systemic dose (see, e.g., Medical Applications of Controlled Release, 1984, Langer and Wise (eds.), CRC Pres., Boca Raton, Fla., vol. 2, pp. 115-138).

The T cell compositions of the present invention may also be administered using any number of matrices. Matrices have been utilized for a number of years within the context of tissue engineering (see, e.g., Principles of Tissue Engineering (Lanza, Langer, and Chick (eds.)), 1997. The present invention utilizes such matrices within the novel context of acting as an artificial lymphoid organ to support, maintain, or modulate the immune system, typically through modulation of T cells. Accordingly, the present invention can utilize those matrix compositions and formulations which have demonstrated utility in tissue engineering. Accordingly, the type of matrix that may be used in the compositions, devices and methods of the invention is virtually limitless and may include both biological and synthetic matrices. In one particular example, the compositions and devices set forth by U.S. Pat. Nos. 5,980,889; 5,913,998; 5,902,745; 5,843,069; 5,787,900; or 5,626,561 are utilized. Matrices comprise features commonly associated with being biocompatible when administered to a mammalian host. Matrices may be formed from both natural or synthetic materials. The matrices may be non-biodegradable in instances where it is desirable to leave permanent structures or removable structures in the body of an animal, such as an implant; or biodegradable. The matrices may take the form of sponges, implants, tubes, telfa pads, fibers, hollow fibers, lyophilized components, gels, powders, porous compositions, or nanoparticles. In addition, matrices can be designed to allow for sustained release seeded cells or produced cytokine or other active agent. In certain embodiments, the matrix of the present invention is flexible and elastic, and may be described as a semisolid scaffold that is permeable to substances such as inorganic salts, aqueous fluids and dissolved gaseous agents including oxygen.

A matrix is used herein as an example of a biocompatible substance. However, the current invention is not limited to matrices and thus, wherever the term matrix or matrices appears these terms should be read to include devices and other substances which allow for cellular retention or cellular traversal, are biocompatible, and are capable of allowing traversal of macromolecules either directly through the substance such that the substance itself is a semi-permeable membrane or used in conjunction with a particular semi-permeable substance.

In certain embodiments of the present invention, cells activated and expanded using the methods described herein, or other methods known in the art where T cells are expanded to therapeutic levels, are administered to a patient in conjunction with (e.g., before, simultaneously or following) any number of relevant treatment modalities, including but not limited to treatment with agents such as antiviral therapy, cidofovir and interleukin-2, Cytarabine (also known as ARA-C) or natalizumab treatment for MS patients or efalizumab treatment for psoriasis patients or other treatments for PML patients. In further embodiments, the T cells of the invention may be used in combination with chemotherapy, radiation, irrimunosuppressive agents, such as cyclosporin, azathioprine, methotrexate, mycophenolate, and FK506, antibodies, or other immunoablative agents such as CAM PATH, anti-CD3 antibodies or other antibody therapies, cytoxin, fludaribine, cyclosporin, FK506, rapamycin, mycophenolic acid, steroids, FR901228, cytokines, and irradiation. These drugs inhibit either the calcium dependent phosphatase calcineurin (cyclosporine and FK506) or inhibit the p70S6 kinase that is important for growth factor induced signaling (rapamycin). (Liu et al., Cell 66:807-815, 1991; Henderson et al., Immun. 73:316-321, 1991; Bierer et al., Curr. Opin. Immun. 5:763-773, 1993; Isoniemi (supra)). In a further embodiment, the cell compositions of the present invention are administered to a patient in conjunction with (e.g., before, simultaneously or following) bone marrow transplantation, T cell ablative therapy using either chemotherapy agents such as, fludarabine, external-beam radiation therapy (XRT), cyclophosphamide, or antibodies such as OKT3 or CAMPATH. In another embodiment, the cell compositions of the present invention are administered following B-cell ablative therapy such as agents that react with CD20, e.g., Rituxan. For example, in one embodiment, subjects may undergo standard treatment with high dose chemotherapy followed by peripheral blood stem cell transplantation. In certain embodiments, following the transplant, subjects receive an infusion of the expanded immune cells of the present invention. In an additional embodiment, expanded cells are administered before or following surgery.

The dosage of the above treatments to be administered to a patient will vary with the precise nature of the condition being treated and the recipient of the treatment. The scaling of dosages for human administration can be performed according to art-accepted practices. The dose for CAMPATH, for example, will generally be in the range 1 to about 100 mg for an adult patient, usually administered daily for a period between 1 and 30 days. The preferred daily dose is 1 to 10 mg per day although in some instances larger doses of up to 40 mg per day may be used (described in U.S. Pat. No. 6,120,766).

### EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### Example 1: Substrate Rigidity Regulates T Cell Activation and Proliferation

The experiments disclosed herein were conducted to evaluate the effects of mechanical properties in a culture substrate on influencing the adhesion, proliferation, and differentiation of different cell types. The impact of substrate stiffness on T cell activation and ex vivo polyclonal expansion using substrates with variable rigidity manufactured from poly(dimethylsiloxane) (PDMS), a biocompatible silicone elastomer was explored. The results presented here demonstrate that the expansion of T cells increases 4-fold following a single round of stimulation on softer substrates. The culturing of naïve CD4+ T cells on softer substrates yielded a 3-fold greater proportion of IFN-γ producing T_{H}1-like cells. These findings reveal that controlling the rigidity of the substrate used to immobilize T cell stimulatory ligands can enhance T cell culture systems. Without wishing to be bound by any particular theory, it is believed that controlling the rigidity of the substrate also may impact the signaling transduction pathways in the T cells.

The materials and methods employed in these experiments are now described.

### Materials and Methods

Fabrication of Silicone-Based Culture Surfaces

PDMS surfaces were fabricated by mixing dimethylsiloxane monomer (Dow Corning Sylgard 184) with its corresponding cross-linking agent according to manufacturer instructions. The ratio of crosslinking agent to base polymer was varied from 1:5 to 1:50. PDMS elastomer slabs of >1 mm were cured at 60° C. for 2 hours prior in multiwall plates to use in T cell culture experiments. Young's Modulus (E) of PDMS prepared at each ratio of crosslinking agent to elastomer base was estimated using a custom-built indentation apparatus. Slabs of PDMS (32 mm×43 mm) with a thickness of ˜10 mm were deformed using a flat, cylindrical head, which makes a no-slip contact with the PDMS surface. A calibrated mass was applied to this head, producing a deflection of the PDMS slab. Hertzian contact between the head and PDMS was assumed (Sneddon et al., 1965, Int J Eng Sci. 3:47-57), which allows estimation of the material's Young's modulus from the head diameter (D), deflection (h), weight (m), gravitational constant (g), and Poisson ratio (ν), assumed to be 0.5 for PDMS using the following equation:

E=(1−ν²)*m*g/(D*h)

Coating of PDMS Surfaces with Antibodies for T Cell Stimulation

Cured PDMS elastomer was incubated with a goat anti-mouse IgG (Cappel, MP Biomedicals) in PBS overnight at 4° C. Unless otherwise indicated, a concentration of 1 μg/mL was used. Following PBS rinsing, a 1 hour incubation with a blocking buffer containing 5% BSA was performed. After washing, agonist monoclonal antibodies to human CD3ε (5 μg/mL, OKT3, Roche Pharmaceuticals) and human CD28 (5 μg/mL, clone 9.3) were captured by incubation in PBS for 2 hrs followed by washing before use.

Primary Cell Preparation and Cell Culture

Primary peripheral blood lymphocytes were obtained under an IRB-approved protocol. Purified total T cells, CD4+ T cells or CD8+ T cells were isolated using RosetteSep isolation kits (Stem Cell Technologies). Naïve CD4+ T cells were obtained by further depletion of CD45RO+ cells using human CD45RO-specific magnetic microbeads, LD selection columns and a VarioMACS system (Miltenyi Biotec). Lymphocytes were cultured in medium comprised of X-VIVO 15 or RPMI (Lonza) supplemented with 5% human serum (GemCell, West Sacramento, Calif.), 10 mM HEPES, L-glutamine, penicillin G and streptomycin. 4.5 μm beads with immobilized anti-human CD3 and anti-human CD28 were used in some experiments at a ratio of 3 beads to 1 cell. T cells were maintained in culture at a concentration of 0.8-1.0×10⁶ cells/mL by regular counting on a Multisizer III particle counter (Beckman-Coulter). In some experiments, cells were also counted by flow cytometry using CountBright beads (BD Biosciences) and monoclonal antibodies to human CD4 and CD8.

Quantitative Real-Time RT-PCR (qRT-PCR) Analyses for IL-2 mRNA

Total RNA was isolated from cells using an RNeasy kit (Qiagen). cDNA was generated by reverse transcription using a High Capacity cDNA Reverse Transcription kit (Applied Biosystems, Carlsbad, Calif.). cDNA was amplified with a predesigned primer-probe set for hIL-2 (Hs00174114_m1; Applied Biosystems). A β-actin specific primer-probe set (Hs999999 03_m1; Applied Biosystems) was used as a normalization control. qRT-PCR assays were performed on a 7500 Fast Real-Time PCR system thermal cycler (Applied Biosystems) using the comparative Ct model. In experiments where the stability of IL-2 mRNA was evaluated, CD4+ lymphocytes were seeded at 0.5×10⁶ cells/well. After 6 hrs, these cultures were treated with/without cyclosporine A (10⁻⁶ M; Sigma-Aldrich) or Actinomycin D (5 μg/mL, Sigma-Aldrich) to inhibit de-novo IL-2 mRNA transcription. Cells were harvested as indicated, and IL-2 mRNA was determined by qRT-PCR analyses.

Antibodies and Flow Cytometry

At the indicated time following activation, cells were stained with a panel of monoclonal antibodies to CD3, CD4, and CD8, CD45RA, CD45RO, CD62L, CCR7, CD27 and LIVE/DEAD Aqua dye (Invitrogen). Flow cytometry was performed using an LSRII (BD Biosciences, San Jose, Calif.), and data were analyzed using FlowJo software (Treestar, Inc., Ashland, Oreg.). In experiments where proliferation was assessed using CFSE dilution, cells were stained with 5 μM CFSE for 5 min, washed twice and resuspended in culture medium prior to initiation of the culture as indicated. Flow cytometry was performed on cells on day 3, and data were analyzed using the Proliferation module as implemented in FlowJo. Intracellular flow cytometry for IFN-γ was performed by re-stimulation of cells with 10⁻⁸ M PMA (Sigma-Aldrich) and 10⁻⁶ M ionomycin (Sigma-Aldrich). GolgiStop was added to the cultures after 1 hour. After an additional 4 hours of incubation, the cells were fixed and permeabilized using Fix/Perm II buffer (Invitrogen) followed by staining with PE-conjugated anti-IFN-γ.

Statistical Analysis

Student's t test for paired data, Wilcoxon Rank Sum or a one-way analysis of variance (ANOVA) were performed using GraphPad Prism version 4.0a (GraphPad Software Inc.). A p-value of <0.05 was considered statistically significant.

The results of this example are now described.

PDMS as a Substrate with Controllable Rigidity for T Cell Activation and Culture

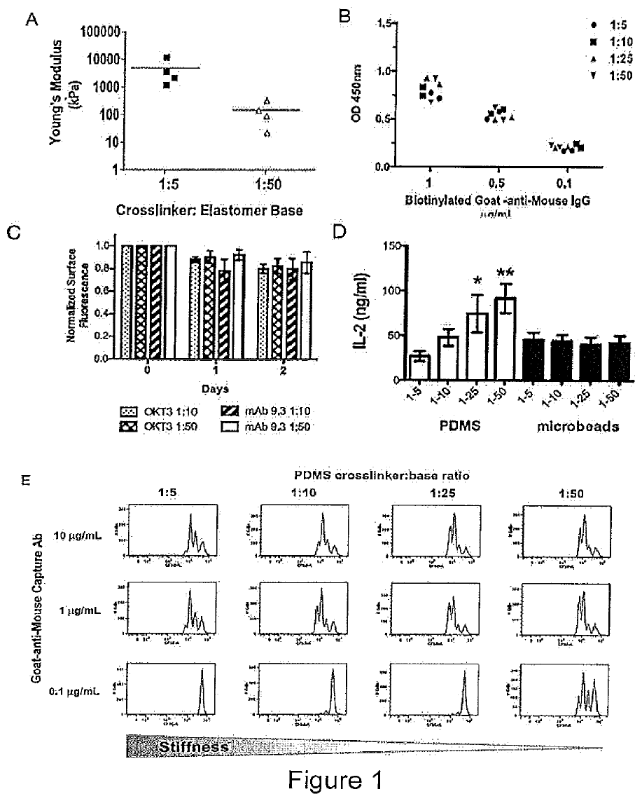

PDMS, a biocompatible organosilicon polymer commonly used as a lubricant, anti-caking agent in foods and anti-bloating agent was selected as a substrate for antibody immobilization. Following crosslinking of the base polymer, PDMS forms an elastomeric material with a highly hydrophobic surface (Androit M, Chao S, Colas A, Cray S, de Buyl F, DeGroot J, et al. Silicones in Industrial Applications. In: Jaeger R D, Gleria M, editors. Inorganic polymers. New York: Nova Science Publishers; 2007. p. 61-161). Proteins, including antibody, passively adsorb to this hydrophobic surface. Alteration of the crosslinking agent to base polymer stoichiometry in the commonly used Sylgard 184 preparation of PDMS provides a simple method for varying the elastic modulus of PDMS from a Young's modulus of >2.3 MPa (stiff) to a range of 50-100 kPa (soft) (FIG. 1A). Prepared this way, this material has been used to study the effects of substrate rigidity on fibroblast focal adhesion formation (Balaban et al., 2001, Nat Cell Biol. 3(5):466-72). Adsorption of anti-CD3 (OKT3) and anti-CD28 (clone 9.3) antibodies to the surface of PDMS provides a system for activation of T cells on substrates with varying elastic modulus, analogous to standard immobilization on more rigid polystyrene tissue culture plastic or glass. Quantitative measurement of enzymatically-coupled primary capture antibody (FIG. 1B) as well as fluorescently-labeled OKT3 and clone 9.3 (data not shown) demonstrate that the amount of antibody adsorbed on PDMS surfaces with varying elastic modulus is equivalent despite changes in the ratio of base polymer to crosslinking agent. Both OKT3 and clone 9.3 also demonstrated stable binding over the course of 48 hours with <20% loss of antibody at 37° C. in complete culture medium independent of the crosslinker ratio (FIG. 1C). Clone 9.3 appeared to demonstrate a slightly more rapid loss from stiff surfaces compared with soft surfaces; however, the quantity of bound clone 9.3 was not significantly different between the PDMS surfaces at 48 h, after which T cells typically transfer to uncoated culture vessels for log-phase ex vivo expansion using planar-activating substrates.

Initial evaluation of T cell activation demonstrated that softer PDMS stimulatory substrate increased IL-2 secretion (FIG. 1D). Since the stiffer PDMS substrates contain more crosslinking agent, the possibility that one of the components in the crosslinking agent may be toxic leading to non-specific inhibition of T cell activation and IL-2 secretion was considered. In order to evaluate this possibility, T cells were simultaneously stimulated with antibody-coated magnetic microbeads in the presence of PDMS with variable rigidity. Unlike T cells stimulated with antibodies immobilized on the PDMS substrate, microbead-stimulated IL-2 secretion was comparable across the different PDMS surfaces arguing against a toxic effect of PDMS elastomer or its crosslinking agent (FIG. 1D).

Since antibody density is an important factor affecting T cell activation and proliferation, the ability of primary human peripheral blood CD4+ T cells to undergo proliferation in response to varying density of OKT3 and clone 9.3 on PDMS surfaces was evaluated. Antibody density-dependent and stiffness-dependent effects on T cell proliferation were observed. Using a carboxyfluoroscein succimidyl ester (CFSE) dilution assay, greater proliferation was reproducibly observed at 72 hours on softer surfaces. The difference in proliferation became more pronounced as the coating concentration of the goat-anti-mouse capture antibody decreased below 1 μg/mL with the proliferation completely lost on harder surfaces coated with antibodies at low concentration (0.1 μg/mL FIG. 1E).

### Polyclonal Expansion of Peripheral Blood T Cells is Enhanced by Culture on Softer Substrates

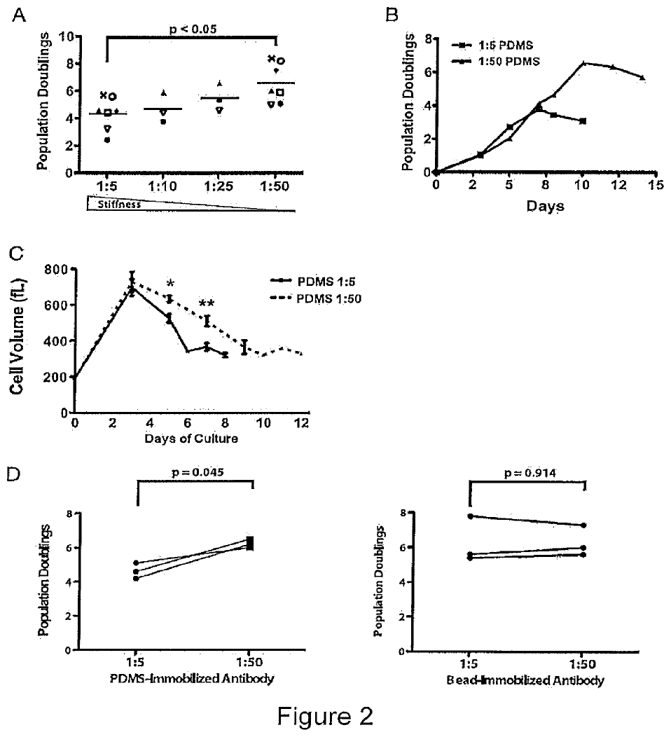

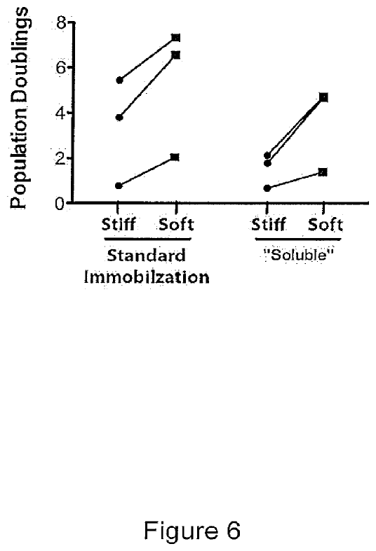

The observation that anti-CD3 and anti-CD28 immobilized on PDMS substrates having a lower elastic modulus stimulate greater IL-2 secretion and short-term proliferation suggested that manipulation of substrate rigidity could be a useful parameter in the ex vivo expansion of T cells. The ability of PDMS substrates to support more long-term proliferation of T cells was therefore evaluated. A graded increase in overall polyclonal expansion of naïve CD4+ T cells across PDMS substrates with increasing Young's elastic modulus was observed (FIG. 2A). On average, a 4-fold increase in overall culture yield is observed when comparing the softest PDMS substrate (1:50 cross-linker to base ratio) to the stiffest PDMS substrate (1:5 cross-linker to base ratio). The difference in overall expansion is primarily due to a more prolonged log-phase growth rather than a difference in the rate of proliferation (FIG. 2B). This enhanced log-phase expansion correlates with a more prolonged blast-phase as shown by the increase in mean cellular volume during the course of the culture (FIG. 2C). Similar to IL-2 secretion, CD4+ T cells cultured on PDMS surfaces, but activated by anti-CD3 and anti-CD28 immobilized on microbeads rather than PDMS, demonstrate comparable proliferation supporting the non-toxic nature of PDMS and the dependency on PDMS immobilization for the observed enhancement in the T cell proliferative response (FIG. 2D). Experiments were also performed using soluble anti-CD3 and anti-CD28 Abs with goat anti-mouse IgG-coated PDMS surfaces to create an equilibrium-binding state that avoids the loss of Ab from precoated surfaces. A similar ˜4-fold enhanced CD4⁺ T cell expansion was observed, with the softer surfaces supporting an effect of PDMS rigidity rather than differential Ab binding between the PDMS surfaces (FIG. 6).

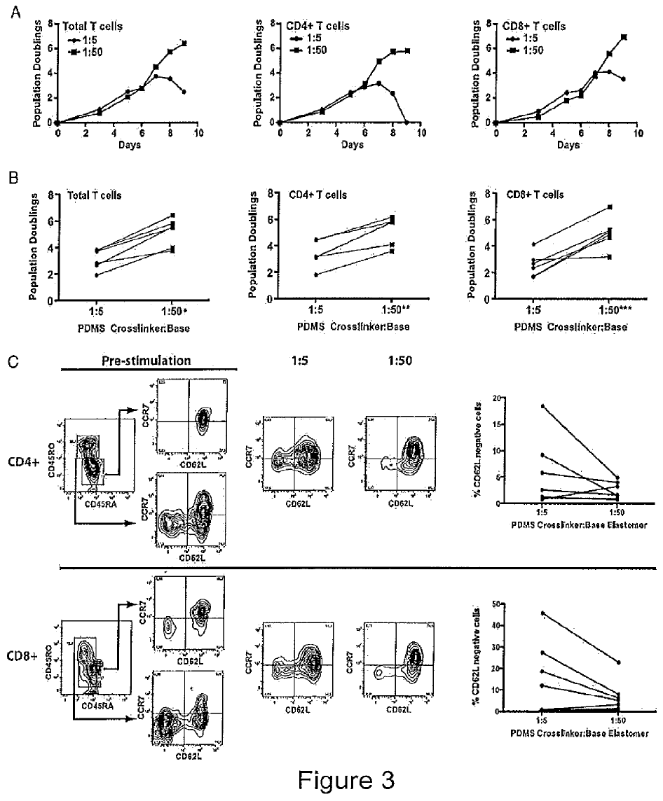

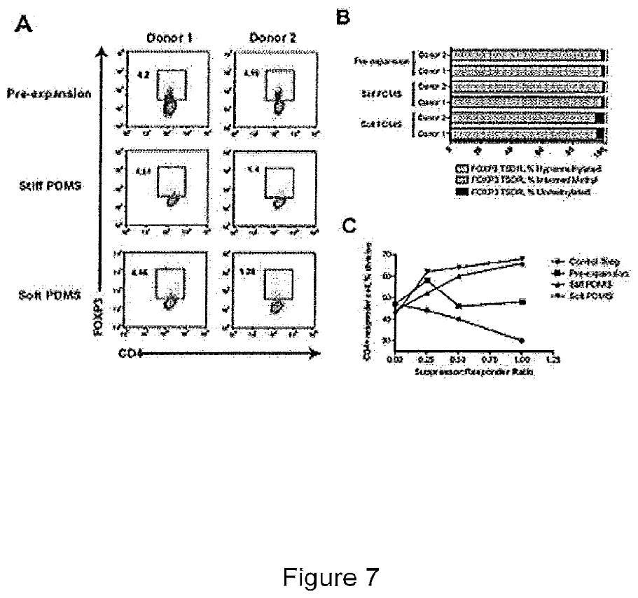

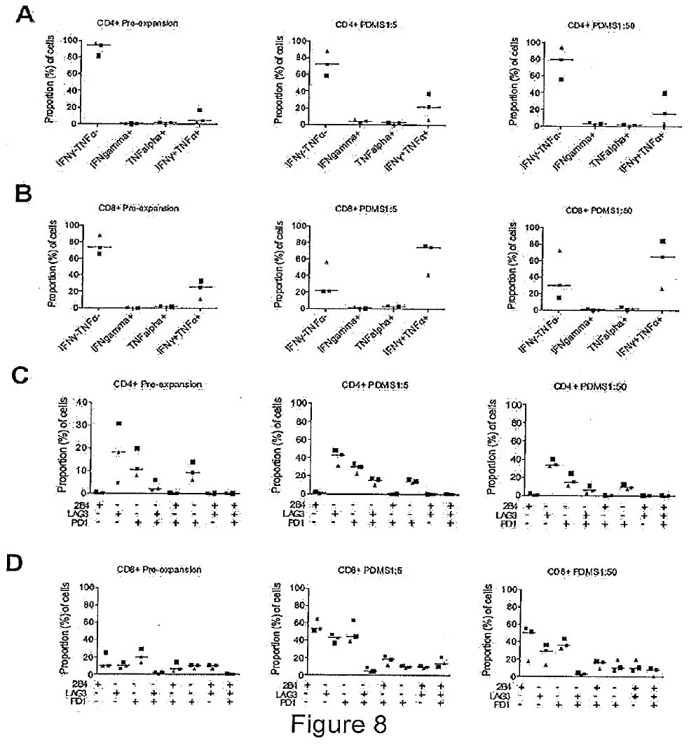

Given the significant enhancement in polyclonal expansion of naïve CD4+ T cells, the ex vivo expansion of a mixed population of CD4+ and CD8+ peripheral blood T cells, a population of cells that is more reflective of the starting population currently being employed for many adoptive immunotherapy studies was evaluated. Consistently, the softer surface also supported an average 4-fold increase in overall T cell expansion compared with stiffer substrate (FIGS. 3A and 3B). This effect was observed in both CD4+ and CD8+ T cells. Since the increase in expansion for a mixed population of cells could be explained by differential expansion of individual T cell subsets, the surface phenotype of cells prior to expansion and at the end of the log-phase of expansion was evaluated. Table 1 shows that the ratio of CD4 to CD8 T cells were comparable between both conditions of rigidity following expansion. The surface expression of CD62L, a cell surface marker routinely used to distinguish memory from effector T cell subsets also exhibited a trend (non-significant, p=0.0745 for CD8+ T cells and p=0.1821 for CD4+ T cells) toward more CD62L negative cells following expansion on stiffer surfaces as shown in FIG. 3C. The phenotypic and functional characteristics of T cells expanded on soft and stiff PDMS surfaces were further evaluated to examine regulatory T cells, effector function, and T cell exhaustion. Neither T cells derived from cultures on soft or stiff PDMS surfaces exhibit a regulatory phenotype based upon flow cytometric markers of regulatory T cells (FIG. 7). The T regulatory cell-specific demethylated region in the FOXP3 locus promoter was hypomethylated following expansion of CD4⁺ T cells on both PDMS surfaces. CD4⁺CD25⁺ and CD4⁺CD25⁻ T cells expanded from either PDMS surfaces also failed to suppress conventional T cell proliferation when cocultured with PBMCs from a separate donor. Although the effector phenotype of the T cells was highly variable between donors, the frequency of cells expressing IFN-γ and TNF-α following activation (FIG. 8A and FIG. 8B) and perforin (<5% positive in CD4⁺ T cells and >70% positive in CD8⁺ T cells, data not shown) is largely similar between cells cultured on soft and stiff surfaces. The cells also showed similar low expression of markers of T cell exhaustion, including 2B4, Lag-3, and PD-1 (Supplemental FIG. 3C, 3D). These findings show that a simple decrease in elastic modulus of the substrate used to immobilize T cell activating antibodies can significantly enhance the T cell activation and proliferative response to these signals. Furthermore, the effects of stiffness are not subset dependent with similar effects observed in both CD4+ and CD8+ T cells.

TABLE 1
Phenotype analysis of mixed lymphocytes following
expansion of stimulatory PDMS surfaces

| | Start of culture | | PDMS 1:5 | | PDMS 1:50 | |
|---|---|---|---|---|---|---|
| | CD4+ | CD8+ | CD4+ | CD8+ | CD4+ | CD8+ |
| Donor | (%) | (%) | (%) | (%) | (%) | (%) |
| 1 | 44.2 | 53.7 | 25.5 | 70.0 | 56 | 43.7 |
| 2 | 65.9 | 33.8 | 37.6 | 62.4 | 45.4 | 53.5 |
| 3 | 46.6 | 49.5 | 27.6 | 70.7 | 20.3 | 78.9 |
| 4 | 65.5 | 29.5 | 72.1 | 7.59 | 81 | 17.9 |
| 5 | 73.7 | 25.4 | 90.5 | 9.39 | 83 | 16.8 |
| 6 | 67.8 | 31.8 | 82.4 | 17.3 | 78.8 | 20.7 |

### Softer Substrates Stimulate Greater IL-2 Secretion Primarily Through Enhanced Transcription

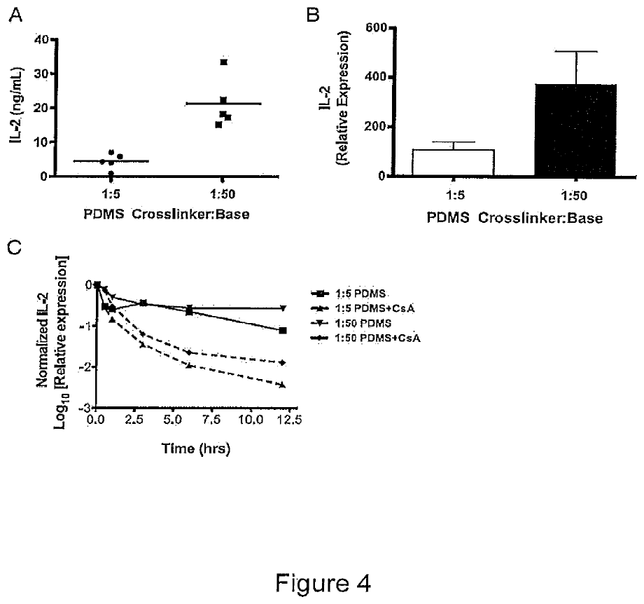

Autocrine production of IL-2 promotes clonal expansion of T cells. Correlating with the enhanced proliferation observed in T cells stimulated with softer substrates, a 4-fold increase in IL-2 production between mixed T cells stimulated on softer compared with stiffer substrates was observed (FIG. 4A). The regulation of IL-2 gene expression is well described to occur at both the level of transcriptional activation and post-transcriptional RNA stability (Lindsten et al., 1989, Science 244(4902):339-43). Experiments were therefore performed to investigate the effect of substrate rigidity on IL-2 mRNA expression and stability. To address IL-2 mRNA stability, experiments were performed to take advantage of the specific inhibition of IL-2 transcription afforded by the calcineurin inhibitor, cyclosporine A (CsA) (Clipstone and Crabtree, 1992, Nature 357(6380):695-7) in addition to the less specific transcriptional inhibitor, actinomycin D. While the expression of IL-2 mRNA was significantly higher at 6 hours on softer substrates (FIG. 4B), the kinetics of mRNA decay following transcriptional arrest by CsA were strikingly similar on both soft and rigid substrates (FIG. 4C). Comparable results were also obtained with actinomycin D (data not shown). These findings indicate that the enhanced IL-2 secretion by T cells stimulated on softer substrates is primarily due to enhanced transcription at the IL-2 gene rather than a difference in the post-transcriptional regulation of IL-2 mRNA stability.

### Softer Substrates Enhance Naïve CD4+ T Cell Th1-Like Differentiation

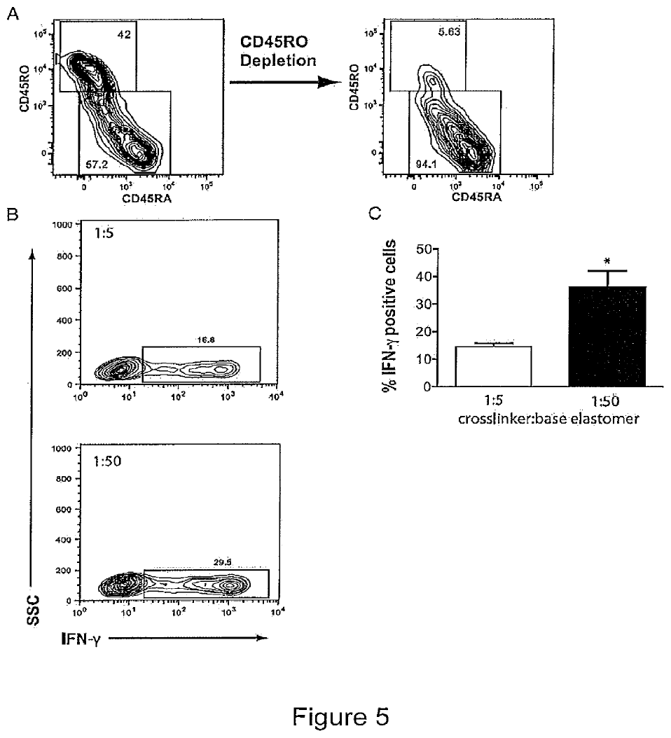

The impact of softer substrates on T cell activation and proliferation lead to considering whether substrate rigidity might also alter CD4+ T cell differentiation since the “strength” of the TCR signal has been reported to affect helper T cell differentiation. The anti-CD3 and anti-CD28 conditions of activation used in the present study have been reported to generate cells of a primarily Th1-like phenotype (Levine et al., 1997, J Immunol 159(12):5921-30). Experiments were therefore performed to evaluate the frequency of IFN-γ producing T cells following expansion of naïve CD4+ T cells. Naïve CD4+ T cells isolated by magnetic bead separation were highly enriched for cells with a CD45RA+, CD62L+ phenotype (FIG. 5A). Following expansion on either the soft or stiff surfaces, the cells exhibited comparable proportions of CD62L+ and CCR7+ cells (data not shown); however, the cells expanded on soft surfaces exhibited a 3-fold increase in the proportion of cells capable of producing IFN-γ (FIGS. 5B and 5C). Combined with the observed enhancement in expansion, softer surfaces produce an ˜1-log greater number of Th1-differentiated IFN-γ producing T cells. Greater than 80% of T cells produce IFN-γ independent of culture surface in the presence of exogenous IL-12 demonstrating that T cells expanded on stiff substrates are capable of differentiation (data not shown). Based upon these results, it was concluded that substrate rigidity might be a useful parameter for controlling CD4+ T cell differentiation, particularly in mixed T cell cultures where the cytokine milieu may be difficult to control.

### Effects of Tunable Rigidity on Ex Vivo T Cell Expansion and Differentiation

The results presented herein is the first known study to demonstrate that the elastic modulus of a cell culture surface influences the activation, proliferation, and differentiation of T cells in ways that could be advantageous for adoptive immunotherapy. Previous studies established that T cells discriminate between surface-bound TCR/CD3 complex ligands and their corresponding soluble forms (Geppert and Lipsky, 1987, J Immunol. 138(6):1660-6; Ma et al., 2008, PLoS Biol 6(2):e43). The results presented herein extends the current body of knowledge, providing evidence that the mechanical properties of the underlying surface used for immobilization is also important, potentially modifying the forces that are generated by T cells and/or the forces sensed within the immune synapse affecting the T cell activation process.

PDMS was selected for the present studies due to its established biocompatibility and stability along with the ease by which its mechanical properties can be modified through variation of the crosslinker to base polymer ratio. It is possible that additional material properties of PDMS change with the variation in crosslinker ratio. Protein adsorption to a surface is highly dependent upon both the hydrophobicity and electrostatic properties of the surface (Nakanishi et al., 2001, J Biosci Bioeng 91(3):233-44). PDMS is well known to be highly hydrophobic with a surface hydrophobicity, as measured by the contact angle of water, that changes little with variation in the crosslinker to base polymer ratio (Mata et al., 2005, Biomed Microdevices 7(4):281-93). Comparable antibody adsorption and stability across the crosslinker to base ratios used was demonstrated in the present study, which is largely consistent with other studies of PDMS surface hydrophobicity and passive adsorption of protein (Gray et al., 2003, J Biomed Mater Res A 66(3):605-14). Brown et al., using a layer-by-layer polyanion coating on PDMS with variable stiffness reported some differences in surface roughness and water contact angle following their surface treatment. These differences appeared to impact vascular smooth muscle cell spreading in the absence of serum; however, cell spreading was similar when serum was present (Brown et al., 2005, Biomaterials 26(16):3123-9).

Physical forces at the TCR/CD3 complex have been linked to intracellular signaling events. Dynamic imaging studies of T cells interacting with supported planar bilayers and solid glass supports demonstrate that TCR microclusters and signaling complexes initially form at the periphery of the immune synapse (Campi et al., 2005, J Exp Med 202(8):1031-6). This peripheral area of the immune synapse is rich in actin and myosin with force exerted on many adhesive receptor-ligand interactions by actin-driven, lamellipodial extension of the T cell membrane as well as myosin-driven contraction of the actin network (Sims et al., 2007, Cell 129(4):773-85). The importance of actin-driven force in the generation of TCR signal transduction is highlighted by studies demonstrating that inhibition of actin polymerization affects T cell activation (Ma et al., 2008, PLoS Biol 6(2):e43; Campi et al., 2005, J Exp Med 202(8):1031-6; Rivas et al., 2004, Mol Cell Biol 24(4):1628-39; Bunnell et al., 2001, Immunity 14(3):315-29). Inhibition of myosin light chain kinase by blebbistatin or the depletion of myosin IIA has also been demonstrated to severely inhibit T cell receptor signaling supporting a critical role for cell-generated force in the T cell activation process (Ilani et al., 2009, Nat Immunol 10(5):531-9).

Changes in the stiffness of the TCR/CD3 ligand support substrate would be expected to dampen the forces applied to individual adhesive receptor-ligand interactions. The range of force necessary to induce signal transduction by the TCR compared with the force necessary to disrupt the mechanical linkage between the receptor and its ligand is currently unknown; however, bond strength and bond lifetime change with the application of a loading force (Evans, 2001, Annu Rev Biophys Biomol Struct 30:105-28). While the bond between avidin and biotin (K_{d} of ˜10⁻¹⁵) is one of the strongest non-covalent associations with a lifetime (1/k_{off}) on the order of ˜10⁹ seconds, this bond's lifetime is reduced dramatically under load to <1 sec with a pN-range load (Merkel et al., 1999, Nature 397(6714):50-3). Much lower forces are therefore expected to have significant effects on the lifetime of bonds between antibodies or TCRs and their ligands, which are several orders of magnitude weaker in their binding affinity. While the softer surfaces used in the present studies have sufficient stiffness to trigger TCR signaling, the net effect of the softer surface may be to provide prolongation of receptor-ligand binding and signaling leading to more effective stimulation.

While mechanical force appears to play a central role in the activation and maintenance of TCR signal transduction, it may also modify downstream signal transduction by the TCR and other receptors affecting immune synapse formation and stability. Focal adhesions (FA) in cells such as fibroblasts depend upon mechanical tension applied to adhesion sites for their assembly (Balaban et al., 2001, Nat Cell Biol. 3(5):466-72; Choquet et al., 1997, Cell 88(1):39-48; Riveline et al., 2001, J Cell Biol 153(6):1175-86; Wolfenson et al., 2011, J Cell Sci 124(Pt 9):1425-32). Many of the same proteins that regulate FA assembly, such as Pyk2, FAK, p130Cas, paxillin, vinculin and talin are also present within T cells at the immunological synapse (IS) (Berg and Ostergaard, 1997, J Immunol 159(4):1753-7; Simonson et al., 2006, J Immunol 177(11):7707-14; Nolz et al., 2007, Mol Cell Biol 27(17):5986-6000; Robertson et al., 2005, J Immunol 175(12):8138-45; Collins et al., 2010, Mol Immunol 47(9):1665-74). The presence of these proteins and other mechano-sensitive proteins within the IS provide mechanism(s) by which forces at the synapse, modified by the substrate rigidity, could influence T cell signal transduction, synapse formation and stability and the activation process.

It is possible that the observed effect of modifying the rigidity of T cell activating substrates could also be relevant in vivo. Cytoskeletal changes within dendritic cells are reported to alter the process of T cell activation (Aldinucci et al., 2010, J Immunol 185(9):5102-10; Al-Alwan et al., 2001, J Immunol 166(3):1452-6; Al-Alwan et al., 2003, J Immunol 171(9):4479-83; Benvenuti et al., 2004, Science 305(5687):1150-3). MHC molecules and co-stimulatory ligands such as CD80 also appear to be anchored to the dendritic cell cytoskeleton (Doty and Clark, 1996, J Immunol 157(8):3270-9; Tseng et al., 2005, J Immunol 175(12):7829-36). Since the actin cytoskeleton represents a gel with viscoelastic properties not unlike PDMS, modification of the cytoskeleton may be a mechanism used by dendritic cells to alter synapse formation, dynamics and ultimately the T cell activation process. Cells attached to solid substrates assume a cytoskeletal rigidity that is proportional to the rigidity of their attachment substrate (Tee et al., 2011, Biophys J 100(5):L25-7). Without wishing to be bound by any particular theory, it is believed that the pliancy and tension of the surrounding ECM within a lymph node likely change quite dramatically during the course of an immune response as the lymph node undergoes marked hyperplasia and distention. Studies can be designed to evaluate the dynamic mechanical properties of the ECM within a lymph node or lymphoid tissue during an immune response.

The observation that substrate rigidity affects the TH-cell differentiation of T cells could be explained by a number of factors. Although cytokines certainly play a critical role, differentiation of TH-cells towards different fates also depends upon TCR ligand density and duration of signaling (Constant et al., 1995, J Exp Med 182(5):1591-6; Hosken et al., 1995, J Exp Med 182(5):1579-84; Rogers et al., 1999, J Immunol 163(3):1205-13; Rogers et al., 1998, J Immunol 161(8):3844-52). Cell-ECM interactions have also been linked to control of gene expression in cells. The PDZ domain containing transcription factors YAP and TAZ were recently demonstrated to be important nuclear mediators of ECM stiffness induced mesenchymal cell differentiation (Dupont et al., 2011, Nature 474(7350):179-83). These pathways or other mechanosensitive pathways could affect lymphocyte differentiation in ways that have not been previously considered, and may depend upon the mechanical properties of culture systems employed to study T cells.

In addition to the fundamental importance of the present findings to the basic study of T cells, a culture system with altered mechanical properties has useful applications to the field of adoptive T cell immunotherapy. Anti-CD3 and anti-CD28 antibodies immobilized either on planar plastic surfaces or plastic microbeads are a commonly employed system for activating T cells ex vivo (Kruisbeek A, Shevach E, Thornton A. Proliferative Assays for T Cell Function. In: Coligan J E, editor. Current protocols in immunology. New York: John Wiley and Sons. p. v. (loose leaf); Levine et al., 1997, J Immunol 159(12):5921-30). Efficient expansion of T cells, especially from patients with cancer, represents a significant challenge. The observation of enhanced expansion on softer surfaces with retention of a mostly CD62L″ memory-like phenotype suggests that a softer substrate might increase the feasibility of adoptive immunotherapy for more patients, especially in challenging diseases like leukemia where few peripheral blood T cells are available in the circulation for expansion. The increased frequency of IFN-γ producing cells observed with softer substrates also suggests that T cells expanded on a softer surface may have improved function following adoptive therapy in cancer. Th1-differentiated, IFN-γ producing cells have been shown in pre-clinical immunotherapy models to be important for efficacy (Nishimura et al., 2000, Cancer Chemother Pharmacol 46 Suppl:552-61).

In summary, these data highlight a novel role for the elastic modulus of a T cell culture surface, a previously unrecognized culture parameter for lymphocytes. Using PDMS elastomers, the results demonstrate that stimulatory substrate rigidity can be controlled to effect changes in T cells of biologic importance. Although not directly evaluated in this study, these data also support the role of force in T cell activation by their antigen receptor. The results presented herein provide evidence that PDMS, a biocompatible polymer, could be used as a platform for the ex vivo culture of T cells for adoptive immunotherapy with potential advantages over currently used rigid plastic surfaces.

### Example 2: Microbeads with Tunable Rigidity Induce T Cell Expansion

The data presented herein demonstrate that substrate materials exhibiting different mechanical rigidities can be formed into geometries other than a planar geometry and which, in some aspects, have several advantages in cell expansion. The data presented herein focus on a spherical bead format, which provides a larger surface area for cell stimulation than the planar format. The spherical bead format has been implemented using conventional, stiff polystyrene beads for cell expansion, but these current systems do not incorporate material rigidity as a key parameter linked to cell expansion. Building upon the data presented elsewhere herein, the data presented herein adapt the PDMS materials into this bead format, and demonstrate effective expansion of primary T cells using this system. While the bead format is the specific implementation of this example, it is clear that the skilled artisan could form other formats, including porous foams and entangled threads, using similar techniques. Similarly, while these results are demonstrated with a specific PDMS formulation, it is envisioned that alternative compositions can be processed in a similar manner, using mechanical rigidity as a key parameter for controlling T cell expansion.

Bead Fabrication

PDMS beads were fabricated using an emulsion technique, applied to the starting materials. Different formulations of Sylgard 184 PDMS, in which the ratio of crosslinker to elastomer base were modified to produce surfaces of different rigidity as described elsewhere herein, were prepared for curing. These uncured materials were added at a 1:5 volume ratio to an aqueous solution of 5% serum albumin in buffer, shaken vigorously, and cured overnight at 56° C. with agitation. This method provided a range of micron-scale beads, from which a population measuring 28-40 microns in diameter are isolated via sequential sieving. The antibody-binding component Protein A was attached to the albumin surface of the beads following curing using a 7% solution of glutaraldehyde. Activating antibodies to CD3 and CD28 were then captured onto the Protein A beads, and the system was used to expand human CD4+ T cells.

T Cell Expansion

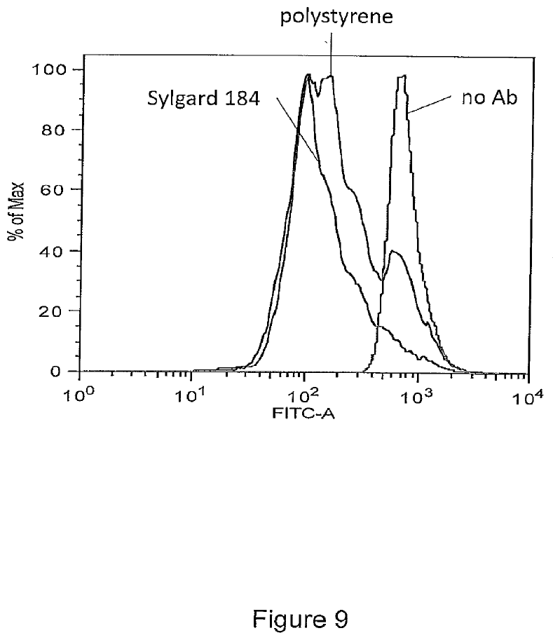

Resting human CD4⁺ T-cells were isolated from whole blood collected via venipuncture using a negative isolation kit (Invitrogen), loaded with 5 μM CFDA-SE for 5 minutes, and then washed twice. Cells were incubated in a 1:3 ratio with coated PDMS beads in RPMI 1640 supplemented with 10% FBS. After 72 hours, T-cell activation was measured by flow cytometry using CFDA-SE staining as a marker of proliferation. CFDA concentration per cell could be divided into peaks, with each group of lower intensity (right-shifted peaks in FIG. 9) representing an additional division of the starting population. As FIG. 9 illustrates, PDMS beads provide strong stimulation and division of a starting population of T cells, in fact more effectively than traditional, protein-coated polystyrene.

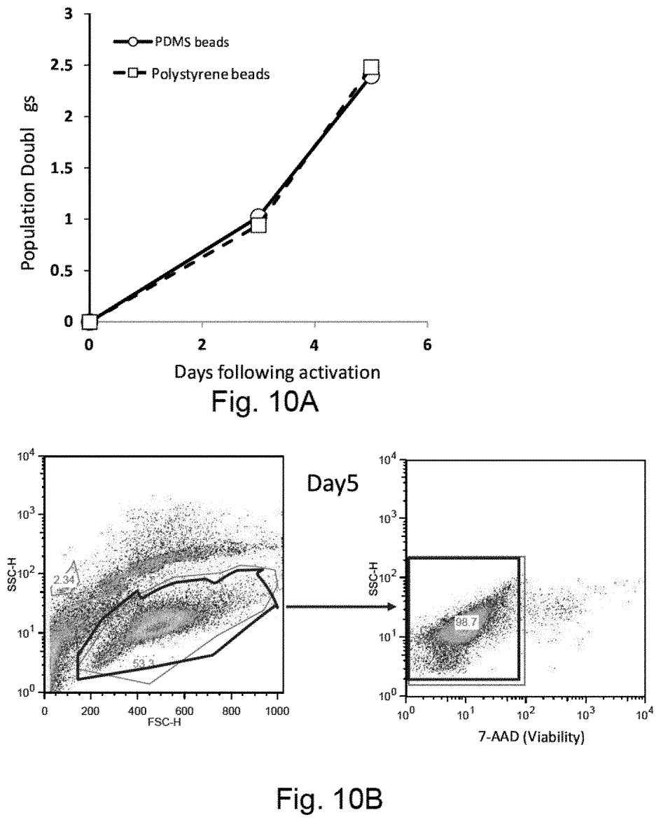

Further studies investigated the extent of CD4+ T cell proliferation when cultured with PDMS beads. As measured by the amount of population doublings, it was observed that CD4+ T cell population could be expanded in culture with PDMS beads (FIG. 10).

These studies are a clear demonstration that materials of varying rigidity can be incorporated into a commercially viable therapeutic platform.

The disclosures of each and every patent, patent application, and publication cited herein are hereby incorporated herein by reference in their entirety.

While the invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A culture system comprising a biocompatible substrate with tunable rigidity for use in stimulating a T cell, wherein said substrate displays on its surface a first agent that stimulates a TCR/CD3 complex-associated signal in T cells and a second agent that stimulates a CD28 accessory molecule on the surface of the T cells.

2. The system of claim 1, wherein said first agent is anti-CD3 antibody.

3. The system of claim 1, wherein said second agent is anti-CD28 antibody.

4. The system of claim 1, wherein said substrate further comprises a co-stimulatory molecule selected from the group consisting of CD80, CD86, 4-1BBL, OX40L, ICOS-L, ICAM, PD-L1 and PD-L2.

5. The system of claim 1, wherein said substrate is a polymer comprising a synthetic polymer or copolymer prepared from at least one of the group of monomers consisting of acrylic acid, methacrylic acid, ethyleneimine, crotonic acid, acrylamide, ethyl acrylate, methyl methacrylate, 2-hydroxyethyl methacrylate, lactic acid, glycolic acid, .ε-caprolactone, acrolein, cyanoacrylate, bisphenol A, epichlorhydrin, hydroxyalkylacrylates, siloxane, dimethylsiloxane, ethylene oxide, ethylene glycol, hydroxyalkyl-methacrylates, N-substituted acrylamides, N-substituted methacrylamides, N-vinyl-2-pyrrolidone, 2,4-pentadiene-1-ol, vinyl acetate, acrylonitrile, styrene, p-amino-styrene, p-amino-benzyl-styrene, sodium styrene sulfonate, sodium 2-sulfoxyethyl methacrylate, vinyl pyridine, aminoethyl methacrylates, 2-methacryloyloxy-trimethylammonium chloride, N,N′-methylenebisacrylamide-, ethylene glycol dimethacrylates, 2,2′-(p-phenylenedioxy)-diethyl dimethacrylate, divinylbenzene, and triallylamine, methylenebis-(4-phenyl-isocyanate).

6. The system of claim 1, wherein the substrate comprises polydimethylsiloxane elastomer (PDMS).

7. The system of claim 1, wherein said substrate exhibits an elastic modulus ranging from about 25 kPa to about 2 MPa.

8. The system of claim 1, wherein said system induces at least one selected from the group consisting of the activation, proliferation, and differentiation of T cells.

9. The system of claim 1, wherein said substrate is a planar substrate.

10. The system of claim 1, wherein said substrate is a spherical substrate.

11. The system of claim 1, wherein said substrate is a microbead.

12. A method of stimulating T cells in culture, the method comprising culturing T cells in the presence of a biocompatible substrate with tunable rigidity, wherein said substrate displays on its surface a first agent that stimulates a TCR/CD3 complex-associated signal in T cells and a second agent that stimulates a CD28 accessory molecule on the surface of the T cells.

13. The method of claim 12, wherein said first agent is anti-CD3 antibody.

14. The method of claim 12, wherein said second agent is anti-CD28 antibody.

15. The method of claim 12, wherein said substrate further comprises a co-stimulatory molecule selected from the group consisting of CD80, CD86, 4-1BBL, OX40L, ICOS-L, ICAM, PD-L1 and PD-L2.

16. The method of claim 12, wherein said substrate is a polymer comprising a synthetic polymer or copolymer prepared from at least one of the group of monomers consisting of acrylic acid, methacrylic acid, ethyleneimine, crotonic acid, acrylamide, ethyl acrylate, methyl methacrylate, 2-hydroxyethyl methacrylate, lactic acid, glycolic acid, .ε-caprolactone, acrolein, cyanoacrylate, bisphenol A, epichlorhydrin, hydroxyalkylacrylates, siloxane, dimethylsiloxane, ethylene oxide, ethylene glycol, hydroxyalkyl-methacrylates, N-substituted acrylamides, N-substituted methacrylamides, N-vinyl-2-pyrrolidone, 2,4-pentadiene-1-ol, vinyl acetate, acrylonitrile, styrene, p-amino-styrene, p-amino-benzyl-styrene, sodium styrene sulfonate, sodium 2-sulfoxyethyl methacrylate, vinyl pyridine, aminoethyl methacrylates, 2-methacryloyloxy-trimethylammonium chloride, N,N′-methylenebisacrylamide-, ethylene glycol dimethacrylates, 2,2′-(p-phenylenedioxy)-diethyl dimethacrylate, divinylbenzene, and triallylamine, methylenebis-(4-phenyl-isocyanate).

17. The method of claim 12, wherein the substrate comprises polydimethylsiloxane elastomer (PDMS).

18. The method of claim 12, wherein said substrate exhibits an elastic modulus ranging from about 25 kPa to about 2 MPa.

19-21. (canceled)

22. A culture system comprising a biocompatible substrate with an optimized rigidity for use in stimulating a T cell, wherein said substrate displays on its surface a first agent that stimulates a TCR/CD3 complex-associated signal in T cells and a second agent that stimulates a CD28 accessory molecule on the surface of the T cells.

23-31. (canceled)

32. A method of adoptive immunotherapy comprising obtaining T cells from a subject, stimulating the T cells by culturing the T cells in the presence of a biocompatible substrate with an optimized rigidity, wherein said substrate displays on its surface a first agent that stimulates a TCR/CD3 complex-associated signal in T cells and a second agent that stimulates a CD28 accessory molecule on the surface of the T cells, and administering the stimulated T cells to the subject.

33-41. (canceled)
